SEQUENCE LISTING (44 sequences)

SEQ ID NO: 1 - protein, 5 aa, Artificial Sequence
  Heavy Chain CDR1 Kabat
DYKIH

SEQ ID NO: 2 - protein, 5 aa, Artificial Sequence
  Heavy Chain CDR1 Kabat
DYAIS

SEQ ID NO: 3 - protein, 5 aa, Artificial Sequence
  Heavy Chain CDR1 Kabat
DYYMH

SEQ ID NO: 4 - protein, 5 aa, Artificial Sequence
  Heavy Chain CDR1 Kabat
DYKIS

SEQ ID NO: 5 - protein, 7 aa, Artificial Sequence
  Heavy Chain CDR1 Chothia
GFTFTDY

SEQ ID NO: 6 - protein, 7 aa, Artificial Sequence
  Heavy Chain CDR1 Chothia
GYTFTDY

SEQ ID NO: 7 - protein, 7 aa, Artificial Sequence
  Heavy Chain CDR1 Chothia
GYSFTDY

SEQ ID NO: 8 - protein, 10 aa, Artificial Sequence
  Heavy Chain CDR1 AbM
GFTFTDYKIH

SEQ ID NO: 9 - protein, 10 aa, Artificial Sequence
  Heavy Chain CDR1 AbM
GFTFTDYAIS

SEQ ID NO: 10 - protein, 10 aa, Artificial Sequence
  Heavy Chain CDR1 AbM
GFTFTDYYMH

SEQ ID NO: 11 - protein, 10 aa, Artificial Sequence
  Heavy Chain CDR1 AbM
GYTFTDYYMH

SEQ ID NO: 12 - protein, 10 aa, Artificial Sequence
  Heavy Chain CDR1 AbM
GYSFTDYKIH

SEQ ID NO: 13 - protein, 10 aa, Artificial Sequence
  Heavy Chain CDR1 AbM
GFTFTDYKIS

SEQ ID NO: 14 - protein, 17 aa, Artificial Sequence
  Heavy Chain CDR2 Kabat
YFNPNSGYSTYNEKFKS

SEQ ID NO: 15 - protein, 17 aa, Artificial Sequence
  Heavy Chain CDR2 Kabat
GINPNSGYSTYAQKFQG

SEQ ID NO: 16 - protein, 17 aa, Artificial Sequence
  Heavy Chain CDR2 Kabat
YFNPNSGYSTYAQKFQG

SEQ ID NO: 17 - protein, 17 aa, Artificial Sequence
  Heavy Chain CDR2 Kabat
WINPNSGYSTYAQKFQG

SEQ ID NO: 18 - protein, 17 aa, Artificial Sequence
  Heavy Chain CDR2 Kabat
WINPNSGYSTYSPSFQG

SEQ ID NO: 19 - protein, 17 aa, Artificial Sequence
  Heavy Chain CDR2 Kabat
WINPNSGYSTYNEKFQG

SEQ ID NO: 20 - protein, 17 aa, Artificial Sequence
  Heavy Chain CDR2 Kabat
YFNPNSGYSNYAQKFQG

SEQ ID NO: 21 - protein, 17 aa, Artificial Sequence
  Heavy Chain CDR2 Kabat
YFNPNSGYATYAQKFQG

SEQ ID NO: 22 - protein, 17 aa, Artificial Sequence
  Heavy Chain CDR2 Kabat
YFNPNSGYSTYSPSFQG

SEQ ID NO: 23 - protein, 8 aa, Artificial Sequence
  Heavy Chain CDR2 Chothia
NPNSGYST

SEQ ID NO: 24 - protein, 8 aa, Artificial Sequence
  Heavy Chain CDR2 Chothia
NPNSGYSN

SEQ ID NO: 25 - protein, 8 aa, Artificial Sequence
  Heavy Chain CDR2 Chothia
NPNSGYAT

SEQ ID NO: 26 - protein, 10 aa, Artificial Sequence
  Heavy Chain CDR2 AbM
YFNPNSGYST

SEQ ID NO: 27 - protein, 10 aa, Artificial Sequence
  Heavy Chain CDR2 AbM
GINPNSGYST

SEQ ID NO: 28 - protein, 10 aa, Artificial Sequence
  Heavy Chain CDR2 AbM
WINPNSGYST

SEQ ID NO: 29 - protein, 10 aa, Artificial Sequence
  Heavy Chain CDR2 AbM
YFNPNSGYSN

SEQ ID NO: 30 - protein, 10 aa, Artificial Sequence
  Heavy Chain CDR2 AbM
YFNPNSGYAT

SEQ ID NO: 31 - protein, 11 aa, Artificial Sequence
  Heavy Chain CDR3 Kabat Chothia AbM
LSPGGYYVMDA

SEQ ID NO: 32 - protein, 11 aa, Artificial Sequence
  Light Chain CDR1 Kabat
KASQNINNYLN

SEQ ID NO: 33 - protein, 11 aa, Artificial Sequence
  Light Chain CDR1 Kabat
RASQGINNYLN

SEQ ID NO: 34 - protein, 7 aa, Artificial Sequence
  Light Chain CDR2 Kabat
NTNNLQT

SEQ ID NO: 35 - protein, 8 aa, Artificial Sequence
  Light Chain CDR3 Kabat
LQHNSFPT

SEQ ID NO: 36 - protein, 120 aa, Rattus norvegicus
  MISC_FEATURE: ICR62 VH
QVNLLQSGAALVKPGASVKLSCKGSGFTFTDYKIHWVKQSHGKSLEWIGYFNPNSGYSTYNEKFKSKATLTADKSTDTAYMELTSLTSEDSATYYCTRLSPGGYYVMDAWGQGASVTVSS

SEQ ID NO: 37 - protein, 108 aa, Rattus norvegicus
  MISC_FEATURE: ICR62 VL
DIQMTQSPSFLSASVGDRVTINCKASQNINNYLNWYQQKLGEAPKRLIYNTNNLQTGIPSRFSGSGSGTDYTLTISSLQPEDFATYFCLQHNSFPTFGAGTKLELKRT

SEQ ID NO: 38 - protein, 120 aa, Artificial Sequence [sequence omitted]

SEQ ID NO: 39 - protein, 108 aa, Artificial Sequence
  I-KC VL construct
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRT

SEQ ID NO: 40 - protein, 328 aa, Homo sapiens
  MISC_FEATURE: Ig gamma-1 CH region
TKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 118 aa, Homo sapiens
  MISC_FEATURE: anti-IGF-1R VH
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 42 - protein, 108 aa, Homo sapiens
  MISC_FEATURE: anti-IGF-1R VL
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 43 - protein, 118 aa, Homo sapiens
  MISC_FEATURE: anti-IGF-1R VH
QVQLVESGGGVVQPGRSLRLSCAASGFTFSSYGMHWVRQAPGKGLEWMAIIWFDGSSKYYGDSVKGRFTISRDNSKNTLYLQMNSLRADDTAVYYCARELGRRYFDLWGRGTLVTVSS

SEQ ID NO: 44 - protein, 108 aa, Homo sapiens
  MISC_FEATURE: anti-IGF-1R VL
EIVLTQSPATLSLSPGERATLSCRASQSVSSYLAWYQQKPGQAPRLLIYDASNRATGIPARFSGSGSGTDFTLTISSLEPEDFAVYYCQQRSKWPPWTFGQGTKVEIK

(19) United States
(12) Patent Application Publication
(10) Pub. No.: US 2011/0200595 A1
(43) Pub. Date: Aug. 18, 2011

(54) TREATMENT WITH A HUMANIZED IgG CLASS ANTI EGFR ANTIBODY AND AN ANTIBODY AGAINST INSULIN LIKE GROWTH FACTOR 1 RECEPTOR

(75) Inventors: Christian Gerdes, Erlenbach (CH); Pablo Umaña, Waedenswil (CH)

(73) Assignee: Roche Glycart, Schlieren (CH)

(21) Appl. No.: 13/027,390

(22) Filed: Feb. 15, 2011

(30) Foreign Application Priority Data
Feb. 18, 2010 (EP) 101539914

(51) Int. Cl. A61K 39/395; A61P 35/00

(52) U.S. Cl. 424/133.1

(57) ABSTRACT

The present invention provides a humanized IgG-class anti-EGFR antibody and an anti-IGF-1R antibody for combined use in treating cancer, with or without additional agents or treatments, such as other anti-cancer drugs or radiation therapy. The invention also encompasses a pharmaceutical composition that is comprised of a combination of a humanized IgG-class anti-EGFR antibody and an anti-IGF-1R antibody in a pharmaceutically acceptable carrier.

### FIELD OF THE INVENTION

The present invention is directed to antibodies and pharmaceutical compositions for use in treating cancer. In particular, the present invention is directed to a humanized IgG-class anti-EGFR antibody and an anti-IGF-1R antibody for combined use in the treatment of cancer.

### BACKGROUND

Cancer is a generic name for a wide range of cellular malignancies characterized by unregulated growth, lack of differentiation, and the ability to invade local tissues and metastasize. These neoplastic malignancies affect, with various degrees of prevalence, every tissue and organ in the body.

A multitude of therapeutic agents have been developed over the past few decades for the treatment of various types of cancer. The most commonly used types of anticancer agents include: Microtubule disruptors (e.g. vinca alkaloids such as vinblastine or vincristine, taxanes such as docetaxel or paclitaxel, epothilones such as ixabepilone), antimetabolites (e.g. anti-folates such as methotrexate or aminopterin, anti-purines such as fludarabine, anti-pyrimidines such as fluorouracil, capecitabine or gemcitabine), topoisomerase inhibitors (e.g. camptothecin, irinotecan or etoposide), DNA intercalators (e.g. doxorubicin, daunorubicin, actinomycin, bleomycin), alkylating agents (e.g. cyclophosphamide, chlorambucil, carmustine, nimustine, streptozocin, busulfan, cisplatin, oxaliplatin, triethylenemelamine, dacarbazine) and hormonal therapy (e.g. glucocorticoids, aromatase inhibitors such as tamoxifene, antiandrogens such as flutamide, gonadotropin-releasing hormone (GnRH) analogs such as leuprolide).

More recently, the importance of targeted therapies in cancer therapy has grown. Such substances—either small molecules or biotherapeutics such as antibodies—interfere with specific targets, e.g. cell surface receptors known to promote carcinogenesis and tumor growth.

### Epidermal Growth Factor Receptor (EGFR) and Anti-EGFR Antibodies

Human epidermal growth factor receptor (also known as HER-1 or ErbB-1, and referred to herein as “EGFR”) is a 170 kDa transmembrane receptor encoded by the c-erbB protooncogene, and exhibits intrinsic tyrosine kinase activity (Modjtahedi et al., Br J Cancer 73, 228-235 (1996); Herbst and Shin, Cancer 94, 1593-1611 (2002)). SwissProt database entry number P00533 provides the sequence of EGFR. There are also isoforms and variants of EGFR (e.g., alternative RNA transcripts, truncated versions, polymorphisms, etc.) including but not limited to those identified by SwissProt database entry numbers P00533-1, P00533-2, P00533-3, and P00533-4. EGFR is known to bind ligands including epidermal growth factor (EGF), transforming growth factor-a (TGF-a), amphiregulin, heparin-binding EGF (HB-EGF), betacellulin, and epiregulin (Herbst and Shin, Cancer 94, 1593-1611 (2002); Mendelsohn and Baselga, Oncogene 19, 6550-6565 (2000)). EGFR regulates numerous cellular processes via tyrosine kinase-mediated signal transduction pathways, including, but not limited to, activation of signal transduction pathways that control cell proliferation, differentiation, cell survival, apoptosis, angiogenesis, mitogenesis, and metastasis (Atalay et al , Ann Oncology 14, 1346-1363 (2003); Tsao and Herbst, Signal 4,4-9 (2003); Herbst and Shin, Cancer 94, 1593-1611 (2002); Modjtahedi et al., Br J Cancer 73, 228-235 (1996)).

Overexpression of EGFR has been reported in numerous human malignant conditions, including cancers of the bladder, brain, head and neck, pancreas, lung, breast, ovary, colon, prostate, and kidney (Atalay et al., Ann Oncology 14, 1346-1363 (2003); Herbst and Shin, Cancer 94, 1593-1611 (2002) Modjtahedi et al., Br J Cancer 73, 228-235 (1996)). In many of these conditions, the overexpression of EGFR correlates or is associated with poor prognosis of the patients (Herbst and Shin, Cancer 94, 1593-1611 (2002) Modjtahedi et al., Br J Cancer 73, 228-235 (1996)). EGFR is also expressed in the cells of normal tissues, particularly the epithelial tissues of the skin, liver, and gastrointestinal tract, although at generally lower levels than in malignant cells (Herbst and Shin, Cancer 94, 1593-1611 (2002)).

Various strategies to target EGFR and block EGFR signaling pathways have been reported. Small-molecule tyrosine kinase inhibitors like gefitinib, erlotinib, canertinib/CI-1033, pelitinib/EKB-569, neratinib/HKI-272, lapatinib/GW572016 and others block autophosphorylation of EGFR in the intracellular tyrosine kinase region, thereby inhibiting downstream signaling events (Tsao and Herbst, Signal 4, 4-9 (2003)). Monoclonal antibodies, on the other hand, target the extracellular portion of EGFR, which results in blocking ligand binding and thereby inhibits downstream events such as cell proliferation (Tsao and Herbst, Signal 4,4-9 (2003)).

Several murine monoclonal antibodies have been generated which achieve such a block in vitro and which have been evaluated for their ability to affect tumor growth in mouse xenograft models (Masui et al., Cancer Res 46, 5592-5598 (1986); Masui et al., Cancer Res 44, 1002-1007 (1984); Goldstein et al., Clin Cancer Res 1, 1311-1318 (1995)). For example, EMD 55900 (EMD Pharmaceuticals) is a murine anti-EGFR monoclonal antibody that was raised against the human epidermoid carcinoma cell line A431 and was tested in clinical studies of patients with advanced squamous cell carcinoma of the larynx or hypopharynx (Bier et al., Eur Arch Otohinolaryngol 252, 433-9 (1995)). In addition, the rat monoclonal antibodies ICR16, ICR62, and ICR80, which bind the extracellular domain of EGFR, have been shown to be effective at inhibiting the binding of EGF and TGF-α the receptor (Modjtahedi et al., Int J Cancer 75, 310-316 (1998)). The murine monoclonal antibody (mAb) 425 is another mAb that was raised against the human A431 carcinoma cell line and was found to bind to a polypeptide epitope on the external domain of the human epidermal growth factor receptor (Murthy et al., Arch Biochem Biophys 252, 549-560 (1987)). A potential problem with the use of murine antibodies in therapy is that non-human monoclonal antibodies can be recognized by the human host as foreign proteins; therefore, repeated injections of such antibodies can lead to the induction of immune responses leading to harmful hypersensitivity reactions. For murine monoclonal antibodies, this is often referred to as a Human Anti-Mouse Antibody, or “HAMA”, response, or a Human Anti-Rat Antibody, or “HARA”, response. Additionally, these “foreign” antibodies can be attacked by the immune system of the host such that they are, in effect, neutralized before they reach their target site. Furthermore, non-human monoclonal antibodies (e.g., murine monoclonal antibodies) typically lack human effector functionality, i.e., they are unable to, inter alia, mediate complement dependent lysis or lyse human target cells through antibody dependent cell-mediated toxicity or Fc-receptor mediated phagocytosis.

To circumvent these problems, chimeric, humanized or even fully human antibodies have been developed, in which only the variable domains, the complementarity determining regions (CDRs) or no parts at all, respectively, are of murine origin, while all other parts of the antibody, in particular the Fc region, are of human origin.

For example, IMC-C225/cetuximab (Erbitux®; ImClone) is a chimeric mouse/human anti-EGFR mAb (based on mouse M225 monoclonal antibody, which resulted in HAMA responses in human clinical trials) that has been reported to demonstrate antitumor efficacy in various human xenograft models (Goldstein et al., Clin Cancer Res 1, 1311-1318 (1995); Herbst and Shin, Cancer 94, 1593-1611 (2002)). The efficacy of IMC-C225 has been attributed to several mechanisms, including inhibition of cell events regulated by EGFR signaling pathways, and possibly by increased antibody-dependent cell-mediated cytotoxicity (ADCC) activity (Herbst and Shin, Cancer 94, 1593-1611 (2002)). IMC-C225 was also used in clinical trials, including in combination with radiotherapy and chemotherapy (Herbst and Shin, Cancer 94, 1593-1611 (2002)). Also, U.S. Pat. No. 5,891,996 (Mateo de Acosta del Rio et al.) discusses a mouse/human chimeric antibody, R3, directed against EGFR. A humanized, R3-based antibody, h-R3/nimotuzumab Mateo et al., Immunotechnology 3, 71-81 (1997); Crombet-Ramos et al., Int J Cancer 101, 567-575 (2002), Boland & Bebb, Expert Opin Biol Ther 9, 1199-1206 (2009), is being developed by Oncoscience (Wedel, Germany) for cancer therapy. U.S. Pat. No. 5,558,864 discusses generation of chimeric and humanized forms of the murine anti-EGFR monoclonal antibody (mAb) 425, and a humanized mAb 425-based antibody, EMD72000/matuzumab (Bier et al., Cancer Immunol Immunother 46, 167-173 (1998), Kim, Curr Opin Mol Ther 6, 96-103 (2004)), is being developed by Merck (Darmstadt, Germany) for cancer therapy. Abgenix, Inc. (Fremont, Calif.) develops ABX-EGF/panitumumab for cancer therapy. ABX-EGF is a fully human anti-EGFR mAb (Yang et al., Crit Rev Oncol/Hematol 38; 17-23 (2001)). Another fully human anti-EGFR mAb, 2F8/zalutumumab, has been developed by Genmab Inc. (Princeton, N.J.) (Bleeker et al., J Immunol 173, 4699-4707 (2004), Lammerts van Bueren, Proc Natl Acad Sci USA 105, 6109-6114 (2008)).

### Insulin-Like Growth Factor-1 Receptor (IGF-1R) and Anti-IGF-1R Antibodies

Similar to EGFR, insulin-like growth factor-1 receptor (IGF-1R, EC 2.7.10.1 (former EC 2.7.112), CD221 antigen) belongs to the family of transmembrane protein tyrosine kinases (LeRoith et al., Endocrin Rev 16, 143-163 (1995); and Adams et al., Cell Mol Life Sci 57, 1050-1093 (2000)). IGF-1R binds IGF-1 with high affinity and initiates the physiological response to this ligand in vivo. IGF-1R also binds to IGF-2, however with slightly lower affinity. The IGF-1 system, including IGF-1R, plays an important role during proliferation of (normal and neoplastic) cells. IGF-1R is found on normal human tissues e. g. placenta, prostate, bladder, kidney, duodenum, small bowel, gallbladder, common bile duct, intrahepatic bile duct, bronchi, tonsil, thymus, breast, sebaceous gland, salivary gland, uterine cervix, and salpinx. IGF-1R overexpression promotes the neoplastic transformation of cells and there exists evidence that IGF-1R is involved in malignant transformation of cells and is therefore a useful target for the development of therapeutic agents for the treatment of cancer (Adams et al., Cell Mol Life Sci 57, 1050-1093 (2000)). In addition to promoting transformation and tumor cell growth and survival, IGF-1R also seems to be capable of inducing effects that could influence tumors at later stages of their natural history. It appears that IGF-1R activation can help cancer cells to cope with the relatively hypoxic and nutrient-deprived microenvironment that pertains in large tumors (Peretz et al., Radiat Res 158, 174-180 (2002); Treins et al., Mol Endocrinol 19, 1304-1317 (2005)). Furthermore, IGF-1R activation or overexpression is associated with an increased propensity for invasion and metastasis (Lopez and Hanahan, Cancer Cell 1, 339-353 (2002); Dunn et al., Cancer Res 58, 3353-3361 (1998), Samani et al., Hum Gene Ther 12, 1969-1977 (2001)).

As for EGFR, various approaches can be taken to target IGF-1R. Small molecule IGF-1R tyrosine kinase inhibitors, such as XL228 (Exelixis Inc., South San Francisco, Calif.) or OSI-906 (OSI Pharmaceuticals, Melville, N.Y.) are being developed and investigated for their efficacy in various cancer types. Also monoclonal antibodies targeting IGF-1R are well known in the art and investigated for their anti-tumor effects in vitro and in vivo, as will be further discussed below.

### Antibody Glycosylation

The oligosaccharide component can significantly affect properties relevant to the efficacy of a therapeutic glycoprotein, including physical stability, resistance to protease attack, interactions with the immune system, pharmacokinetics, and specific biological activity. Such properties may depend not only on the presence or absence, but also on the specific structures, of oligosaccharides. Some generalizations between oligosaccharide structure and glycoprotein function can be made. For example, certain oligosaccharide structures mediate rapid clearance of the glycoprotein from the bloodstream through interactions with specific carbohydrate binding proteins, while others can be bound by antibodies and trigger undesired immune reactions (Jenkins et al., Nat Biotechnol 14, 975-81 (1996)).

IgG1 type antibodies, the most commonly used antibodies in cancer immunotherapy, are glycoproteins that have a conserved N-linked glycosylation site at Asn 297 in each CH2 domain. The two complex biantennary oligosaccharides attached to Asn 297 are buried between the CH2 domains, forming extensive contacts with the polypeptide backbone, and their presence is essential for the antibody to mediate effector functions such as antibody dependent cell-mediated cytotoxicity (ADCC) (Lifely et al., Glycobiology 5, 813-822 (1995); Jefferis et al., Immunol Rev 163, 59-76 (1998); Wright and Morrison, Trends Biotechnol 15, 26-32 (1997)).

Cell-mediated effector functions of monoclonal antibodies, such as the anti-EGFR and anti-IGF-1R antibodies mentioned above and below, respectively (e.g. cetuximab, nimotuzumab, panitumumab, cixutumumab, figitumumab), can be enhanced by engineering their oligosaccharide component as described in Umana et al., Nat Biotechnol 17, 176-180 (1999) and U.S. Pat. No. 6,602,684 (WO 99/54342). Umana et al. showed that overexpression of β(1,4)-N-acetylglucosaminyltransferase III (GnTIII), a glycosyltransferase catalyzing the formation of bisected oligosaccharides, in Chinese hamster ovary (CHO) cells significantly increases the in vitro ADCC activity of antibodies produced in those cells. Alterations in the composition of the Asn 297 carbohydrate or its elimination also affect binding of the antibody Fc-domain to FcγR and C1q protein (Umana et al., Nat Biotechnol 17, 176-180 (1999); Davies et al., Biotechnol Bioeng 74, 288-294 (2001); Mimura et al., J Biol Chem 276, 45539-45547 (2001); Radaev et al., J Biol Chem 276, 16478-16483 (2001); Shields et al., J Biol Chem 276, 6591-6604 (2001); Shields et al., J Biol Chem 277, 26733-26740 (2002); Simmons et al., J Immunol Methods 263, 133-147 (2002)).

An anti-neoplastic drug would ideally kill cancer cells selectively, with a wide therapeutic index relative to its toxicity towards non-malignant cells. It would also retain its efficacy against malignant cells, even after prolonged exposure to the drug. Unfortunately, none of the current anti-cancer therapies possess such an ideal profile. Instead, most possess very narrow therapeutic indexes. Furthermore, cancerous cells exposed to slightly sub-lethal concentrations of an anti-neoplastic agent will very often develop resistance to such an agent, and quite often cross-resistance to several other antineoplastic agents as well.

Thus, there is a need for more efficacious treatment for neoplasia and other proliferative disorders. Strategies for enhancing the therapeutic efficacy of existing drugs have involved changes in the schedule for their administration, and also their use in combination with other anticancer or biochemical modulating agents. Combination therapy is well known as a method that can result in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect is synergistic (the efficacy of the combination is greater than the sum of the effects of each drug given alone). For example, when combined with 5-fluorouracil and leucovorin, oxaliplatin exhibits response rates of 25-40% as first-line treatment for colorectal cancer (Raymond, E. et al., Semin Oncol 25(2 Suppl. 5), 4-12 (1998)).

Likewise, the combined use of several antibodies, directed to specific targets on the surface of cancer cells, might increase anti-cancer efficacy, compared to treatment with a single antibody. This is particularly interesting, because antibodies generally provide a better selectivity towards cancer cells than chemotherapeutic drugs, i.e. they have less adverse effects on healthy tissues.

### SUMMARY

One aspect of the invention provides a combination comprising a humanized IgG-class anti-EGFR antibody and an anti-IGF-1R antibody. In one embodiment, the IgG-class anti-EGFR antibody comprises a) in the heavy chain variable domain a CDR1 of SEQ ID NO:1, a CDR2 of SEQ ID NO:16 and a CDR3 of SEQ ID NO:31, and b) in the light chain variable domain a CDR1 of SEQ ID NO:33, a CDR2 of SEQ ID NO:34 and a CDR3 of SEQ ID No:35.

In one embodiment, the humanized IgG-class anti-EGFR antibody is glycoengineered to have an altered oligosaccharide structure in the Fc region. In one embodiment, the humanized IgG-class anti-EGFR antibody has an increased proportion of non-fucosylated oligosaccharides in its Fc-region, compared to a non-glycoengineered antibody. In one embodiment, the anti-IGF-1R antibody is modified to have increased effector function and/or increased Fc receptor binding compared to an unmodified antibody. In one embodiment, the anti-IGF-1R antibody is glycoengineered to have an altered oligosaccharide structure in the Fc region. In one embodiment, the anti-IGF-1R antibody comprises a heavy chain variable domain amino acid sequence of SEQ ID NO:41 or SEQ ID NO:43 and a light chain variable domain amino acid sequence of SEQ ID NO:42 or SEQ ID NO:44.

In one embodiment, the humanized IgG-class anti-EGFR antibody and the anti-IGF-1R antibody are contained in the same formulation. In another embodiment, the humanized IgG-class anti-EGFR antibody and the anti-IGF-1R antibody are contained in different formulations.

Another aspect of the invention provides a pharmaceutical composition comprising a humanized IgG-class anti-EGFR antibody and an anti-IGF-1R antibody in a pharmaceutically acceptable carrier. In one embodiment, the IgG-class anti-EGFR antibody comprises a) in the heavy chain variable domain a CDR1 of SEQ ID NO:1, a CDR2 of SEQ ID NO:16 and a CDR3 of SEQ ID NO:31, and b) in the light chain variable domain a CDR1 of SEQ ID NO:33, a CDR2 of SEQ ID NO:34 and a CDR3 of SEQ ID No:35. In one embodiment, the humanized IgG-class anti-EGFR antibody is glycoengineered to have an altered oligosaccharide structure in the Fc region. In one embodiment, the anti-IGF-1R antibody is modified to have increased effector function and/or increased Fc receptor binding compared to an unmodified antibody. In one embodiment, the pharmaceutical composition further comprises one or more other therapeutically active agents.

Another aspect of the invention provides a method for the treatment of cancer, comprising administering to a subject in need thereof a humanized IgG-class anti-EGFR antibody and an anti-IGF-1R antibody. In one embodiment, the IgG-class anti-EGFR antibody comprises a) in the heavy chain variable domain a CDR1 of SEQ ID NO:1, a CDR2 of SEQ ID NO:16 and a CDR3 of SEQ ID NO:31, and b) in the light chain variable domain a CDR1 of SEQ ID NO:33, a CDR2 of SEQ ID NO:34 and a CDR3 of SEQ ID No:35. In one embodiment, the humanized IgG-class anti-EGFR antibody is glycoengineered to have an altered oligosaccharide structure in the Fc region. In one embodiment, the anti-IGF-1R antibody is modified to have increased effector function and/or increased Fc receptor binding compared to an unmodified antibody.

In one embodiment, the humanized IgG-class anti-EGFR antibody and the anti-IGF-1R antibody are administered in the same formulation. In another embodiment, the humanized IgG-class anti-EGFR antibody and the anti-IGF-1R antibody are administered in different formulations. In one embodiment, the humanized IgG-class anti-EGFR antibody and the anti-IGF-1R antibody are administered by the same route. In another embodiment, the humanized IgG-class anti-EGFR antibody and the anti-IGF-1R antibody are administered by different routes. In one embodiment, one or more other anti-cancer agents are also administered to the subject.

Another aspect of the invention provides a method for manufacturing a medicament, comprising combining a therapeutically effective amount of a humanized IgG-class anti-EGFR antibody and an anti-IGF-1R antibody. In one embodiment, the IgG-class anti-EGFR antibody comprises a) in the heavy chain variable domain a CDR1 of SEQ ID NO:1, a CDR2 of SEQ ID NO:16 and a CDR3 of SEQ ID NO:31, and b) in the light chain variable domain a CDR1 of SEQ ID NO:33, a CDR2 of SEQ ID NO:34 and a CDR3 of SEQ ID No:35.

Yet another aspect of the invention provides a kit comprising a humanized IgG-class anti-EGFR antibody and an anti-IGF-1R antibody, in the same or in separate containers, wherein the IgG-class anti-EGFR antibody comprises a) in the heavy chain variable domain a CDR1 of SEQ ID NO:1, a CDR2 of SEQ ID NO:16 and a CDR3 of SEQ ID NO:31, and b) in the light chain variable domain a CDR1 of SEQ ID NO:33, a CDR2 of SEQ ID NO:34 and a CDR3 of SEQ ID No:35.

### BRIEF DESCRIPTION OF THE FIGURES

FIG. 1. Kaplan-Meier curves representing survival of SCID beige mice bearing A549 lung adenocarcinoma xenografts, treated with vehicle (solid line), 25 mg/kg partially fucosylated hu-ICR62 IgG1 anti-EGFR mAb (dashed/dotted line), 25 mg/kg partially fucosylated hu-ICR62 IgG1 anti-EGFR mAb and 10 mg/kg rhu anti-IGF-1R mAb 18 (dotted line) or 25 mg/kg cetuximab (Erbitux™) and 10 mg/kg rhu anti-IGF-1R mAb 18 (dashed line).

FIG. 2. Kaplan-Meier curves representing survival of SCID beige mice bearing A549 lung adenocarcinoma xenografts, treated with vehicle (solid line), partially fucosylated 25 mg/kg hu-ICR62 IgG1 anti-EGFR mAb (dashed line), 10 mg/kg partially fucosylated rhu anti-IGF-1R mAb 18 (dashed/dotted line), or 25 mg/kg partially fucosylated hu-ICR62 IgG1 anti-EGFR mAb and 10 mg/kg partially fucosylated rhu anti-IGF-1R mAb 18 (dotted line).

### DETAILED DESCRIPTION OF EMBODIMENTS OF THE INVENTION

Recognizing the great therapeutic potential of combining antibodies which target surface receptors on cancer cells involved in cancer progression, the present invention provides a humanized IgG-class anti-EGFR antibody and an anti-IGF-1R antibody for combined use in treating cancer.

The invention also encompasses a pharmaceutical composition, in particular for use in treating cancer, comprising a humanized IgG-class anti-EGFR antibody and an anti-IGF-1R antibody in a pharmaceutically acceptable carrier.

The present invention is further directed to a method for the treatment of cancer comprising administering to a subject in need a humanized IgG-class anti-EGFR antibody and an anti-IGF-1R antibody.

Preferably, a therapeutically effective amount of the combination of the humanized IgG-class anti-EGFR antibody and the anti-IGF-1R antibody is intended for administration to the patient simultaneously or sequentially (in any order), in the same or in different formulations and with or without additional agents or treatments, such as other anti-cancer drugs or radiation therapy.

Preferred humanized IgG-class anti-EGFR antibodies useful for the present invention are described in WO 2006/082515 and WO 2008/017963, the entire content of which is incorporated herein by reference, and include antibodies which are characterized in that they are chimeric antibodies having the binding specificity of the rat monoclonal antibody ICR62 and that their effector functions are enhanced by altered glycosylation.

Preferred anti-EGFR antibodies are characterized in that they comprise at least one (i.e. one, two, three, four, five, or six) complementarity determining region (CDR) of the rat ICR62 antibody, or a variant or truncated form thereof containing at least the specificity-determining residues for said CDR, and comprising a sequence derived from a heterologous polypeptide. By “specificity-determining residue” is meant those residues that are directly involved in the interaction with the antigen. Specifically, preferred anti-EGFR antibodies comprise: (a) a heavy chain CDR1 sequence selected from a group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, and SEQ ID NO:13; (b) a heavy chain CDR2 sequence selected from a group consisting of: SEQ ID NO:14, SEQ ID NO:15, SEQ ID

NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30; and (c) the heavy chain CDR3 sequence SEQ ID NO:31. Preferred anti-EGFR antibodies further comprise: (a) a light chain CDR1 sequence selected from the group consisting of SEQ ID NO:32 and SEQ ID NO:33; (b) the light chain CDR2 sequence SEQ ID NO:34; and (c) the light chain CDR3 sequence SEQ ID NO:35.

More preferred anti-EGFR antibodies are characterized in that they comprise at least three CDRs of the rat ICR62 antibody, or variants or truncated forms thereof containing at least the specificity-determining residues for said CDRs.

Most preferred anti-EGRF antibodies useful for the present invention comprise:

a) in the heavy chain variable domain a CDR1 of SEQ ID NO:1, a CDR2 of SEQ ID NO:16, and a CDR3 of SEQ ID NO:31, and

b) in the light chain variable domain a CDR1 of SEQ ID NO:33, a CDR2 of SEQ ID NO:34, and a CDR3 of SEQ ID NO:35.

The possible CDR sequences of preferred anti-EGFR antibodies useful for the invention are summarized in Table 1 (heavy chain CDRs) and Table 2 (light chain CDRs).

TABLE 1
Heavy chain CDR amino acid sequences
of preferred anti-EGFR antibodies.*

| CDR | | Amino Acid Sequence | SEQ ID NO |
|---|---|---|---|
| Heavy | Kabat | DYKIH | 1 |
| Chain | | DYAIS | 2 |
| CDR1 | | DYYMH | 3 |
| | | DYKIS | 4 |
| | Chothia | GFTFTDY | 5 |
| | | GYTFTDY | 6 |
| | | GYSFTDY | 7 |
| | | GFTFTDYKIH | 8 |
| | AbM | GFTFTDYAIS | 9 |
| | | GFTFTDYYMH | 10 |
| | | GYTFTDYYMH | 11 |
| | | GYSFTDYKIH | 12 |
| | | GFTFTDYKIS | 13 |
| Heavy | Kabat | YFNPNSGYSTYNEKFKS | 14 |
| Chain | | GINPNSGYSTYAQKFQG | 15 |
| CDR2 | | YFNPNSGYSTYAQKFQG | 16 |
| | | WINPNSGYSTYAQKFQG | 17 |
| | | WINPNSGYSTYSPSFQG | 18 |
| | | WINPNSGYSTYNEKFQG | 19 |
| | | YFNPNSGYSNYAQKFQG | 20 |
| | | YFNPNSGYATYAQKFQG | 21 |
| | | YFNPNSGYSTYSPSFQG | 22 |
| | Chothia | NPNSGYST | 23 |
| | | NPNSGYSN | 24 |
| | | NPNSGYAT | 25 |
| | AbM | YFNPNSGYST | 26 |
| | | GINPNSGYST | 27 |
| | | WINPNSGYST | 28 |
| | | YFNPNSGYSN | 29 |
| | | YFNPNSGYAT | 30 |
| Heavy | Kabat | LSPGGYYVMDA | 31 |
| Chain | Chothia | | |
| CDR3 | AbM | | |

TABLE 2
Light chain CDR amino acid sequences
of preferred anti-EGFR antibodies.*

| CDR | Amino Acid Sequence | SEQ ID NO |
|---|---|---|
| Kabat Light | KASQNINNYLN | 32 |
| Chain CDR1 | RASQGINNYLN | 33 |
| Kabat Light | NTNNLQT | 34 |
| Chain CDR2 | | |
| Kabat Light | LQHNSFPT | 35 |
| Chain CDR3 | | |

* “Kabat” refers to the CDRs as defined by
Kabat et al., “Sequences of Proteins of
Immunological Interest”, National Institutes
of Health, Bethesda (1983)
“Chothia” refers to the CDRs as defined by
Chothia et al., J Mol Biol 196, 901-917 (1987)
“AbM” refers to the CDRs as defined by Oxford
Molecular's AbM antibody modeling software

Other preferred anti-EGFR antibodies useful for the present invention comprise the heavy chain variable domain (V_{H}) of the rat ICR62 antibody according to SEQ ID NO:36, or a variant thereof; and a non-murine polypeptide. Further, preferred anti-EGFR antibodies may comprise the light chain variable domain (V_{L}) of the rat ICR62 antibody according to SEQ ID NO:37, or a variant thereof; and a non-murine polypeptide.

More preferred anti-EGFR antibodies useful for the invention comprise the heavy chain variable domain of SEQ ID NO:38 and the light chain variable domain of SEQ ID NO:39.

The heavy and light chain variable domain amino acid sequences of preferred anti-EGFR antibodies are shown in Table 3. The preferred anti-EGFR antibodies useful for the invention may also comprise amino acid sequences of at least 80%, 85%, 90%, 95%, 96%, 97%, 98% or 99% sequence identity to those shown in Table 3, or the amino acid sequences shown in Table 3 with conservative amino acid substitutions.

TABLE 3
Heavy and light chain variable domain amino
acid sequences of preferred anti-EGFR antibodies.

| | | SEQ ID |
|---|---|---|
| CONSTRUCT | AMINO ACID SEQUENCE | NO |
| ICR62 V_{H} | QVNLLQSGAALVKPGASVKLSCKGSGFTFTD | 36 |
| | YKIHWVKQSHGKSLEWIGYFNPNSGYSTYNE | |
| | KFKSKATLTADKSTDTAYMELTSLTSEDSAT | |
| | YYCTRLSPGGYYVMDAWGQGASVTVSS | |
| ICR62 V_{L} | DIQMTQSPSFLSASVGDRVTINCKASQNINN | 37 |
| | YLNWYQQKLGEAPKRLIYNTNNLQTGIPSRF | |
| | SGSGSGTDYTLTISSLQPEDFATYFCLQHNS | |
| | FPTFGAGTKLELKRT | |
| I-HHD V_{H} | QVQLVQSGAEVKKPGSSVKVSCKASGFTFTD | 38 |
| | YKIHWVRQAPGQGLEWMGYFNPNSGYSTYAQ | |
| | KFQGRVTITADKSTSTAYMELSSLRSEDTAV | |
| | YYCARLSPGGYYVMDAWGQGTTVTVSS | |
| I-KC V_{L} | DIQMTQSPSSLSASVGDRVTITCRASQGINN | 39 |
| | YLNWYQQKPGKAPKRLIYNTNNLQTGVPSRF | |
| | SGSGSGTEFTLTISSLQPEDFATYYCLQHNS | |
| | FPTFGQGTKLEIKRT | |

Preferred anti-EGFR antibodies useful for the invention are primatized or, more preferred, humanized antibodies.

Preferably, the anti-EGFR antibodies useful for the invention comprise a human Fc region. More preferably, the human heavy chain constant region is Ig gamma-1, as set forth in SEQ ID NO:40, i.e. the antibody is of human IgG1 subclass.

Human heavy chain constant region Ig gamma-1 amino acid sequence (SEQ ID NO:40):

TKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKA
EPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELT
KNQVSLTCLVKGFYPSDIAVEWESNGQPENNYKTTPPVLDSDGSFFLY
SKLTVDKSRWQQGNVFSCSVMHEALHNHYTQKSLSLSPGK

However, variants and isoforms of the human Fc region are also contemplated. For example, variant Fc regions suitable for use in the present invention can be produced according to the methods taught in U.S. Pat. No. 6,737,056 to Presta (Fc region variants with altered effector function due to one or more amino acid modifications); or in U.S. Patent Appl. Nos. 60/439,498; 60/456,041; 60/514,549; or WO 2004/063351 (variant Fc regions with increased binding affinity due to amino acid modification); or in U.S. patent application Ser. No. 10/672,280 or WO 2004/099249 (Fc variants with altered binding to FcyR due to amino acid modification), the contents of each of which is herein incorporated by reference in its entirety.

In another preferred embodiment, anti-EGFR antibodies useful for the invention have been glycoengineered to have an altered oligosaccharide structure in the Fc region.

Specifically, preferred anti-EGFR antibodies have an increased proportion of non-fucosylated oligosaccharides in the Fc region as compared to non-glycoengineered antibodies. Preferably, the percentage of non-fucosylated oligosaccharides is at least 20%, more preferably at least 50-70%, most preferably at least 75%. Anti-EGFR antibodies useful for the invention having such percentages of non-fucosylated oligosaccharides are further termed as partially fucosylated. The non-fucosylated oligosaccharides may be of the hybrid or complex type.

Preferred anti-EGFR antibodies may also have an increased proportion of bisected oligosaccharides in the Fc region. Preferably, the percentage of bisected oligosaccharides in the Fc region of the antibody is at least 50%, more preferably, at least 60%, at least 70%, at least 80%, or at least 90%, and most preferably at least 90-95% of the total oligosaccharides.

Particularly preferred anti-EGFR antibodies have an increased proportion of bisected, non-fucosylated oligosaccharides in the Fc region. The bisected, non-fucosylated oligosaccharides may be either hybrid or complex. Specifically, anti-EGFR antibodies are preferred in which at least 15%, more preferably at least 20%, more preferably at least 25%, more preferably at least 30%, more preferably at least 35% of the oligosaccharides in the Fc region of the antibody are bisected, non-fucosylated.

Preferred anti-EGFR antibodies are also characterized in that they have been glycoengineered to have increased effector function and/or increased Fc receptor binding affinity.

Preferably, the increased effector function is one or more of the following: increased Fc-mediated cellular cytotoxicity (including increased antibody-dependent cell-mediated cytotoxicity (ADCC)), increased antibody-dependent cellular phagocytosis (ADCP), increased cytokine secretion, increased immune-complex-mediated antigen uptake by antigen-presenting cells, increased binding to natural killer (NK) cells, increased binding to macrophages, increased binding to monocytes, increased binding to polymorphonuclear cells, increased direct signaling inducing apoptosis, increased crosslinking of target-bound antibodies, increased dendritic cell maturation, or increased T cell priming. The increased Fc receptor binding affinity is preferably increased binding to a Fcy activating receptor, most preferably increased binding to FcγRIIIα.

The most preferred IgG-class anti-EGFR antibody useful for the invention is characterized in that it comprises the heavy chain variable domain of SEQ ID NO:38 and the light chain variable domain of SEQ ID NO:39, is humanized, and comprises the human heavy chain constant region Ig gamma-1, as set forth in SEQ ID NO:40. This antibody is termed “hu-ICR62 IgG1 anti-EGFR mAb”. hu-ICR62 IgG1 anti-EGFR mAb may or may not be partially fucosylated, i.e. glycoengineered as described above, to have an increased proportion of non-fucosylated oligosaccharides in the Fc region as compared to non-glycoengineered antibodies.

Other suitable humanized IgG-class anti-EGFR antibodies useful for the invention include also cetuximab/IMC-C225 (Erbitux®, described in Goldstein et al., Clin Cancer Res 1, 1311-1318 (1995)), panitumumab/ABX-EGF (Vectibix®, described in Yang et al., Cancer Res 59, 1236-1243 (1999), Yang et al., Critical Reviews in Oncology/Hematology 38, 17-23 (2001)), nimotuzumab/h-R3 (TheraCim®, described in Mateo et al., Immunotechnology 3, 71-81 (1997); Crombet-Ramos et al., Int J Cancer 101, 567-575 (2002), Boland & Bebb, Expert Opin Biol Ther 9, 1199-1206 (2009)), matuzumab/EMD 72000 (described in Bier et al., Cancer Immunol Immunother 46, 167-173 (1998), Kim, Curr Opin Mol Ther 6, 96-103 (2004)), and zalutumumab/2F8 (described in Bleeker et al., J Immunol 173, 4699-4707 (2004), Lammerts van Bueren, PNAS 105, 6109-6114 (2008)), which may or may not be partially fucosylated, i.e. glycoengineered as described above, to have an increased proportion of non-fucosylated oligosaccharides in the Fc region as compared to non-glycoengineered antibodies.

Suitable anti-IGF-1R antibodies useful for the present invention are described e.g. in Benini et al., Clin Cancer Res 7, 1790-1797 (2001); Soos et al., J Biol Chem 267, 12955-12963 (1992); Li et al., Biochem Biophys Res Commun 196, 92-98 (1993); Delafontaine et al., J Mol Cell Cardiol 26, 1659-1673 (1994); Gustafson and Rutter, J Biol Chem 265, 18663-18667 (1990); Rohlik et al., Biochem Biophys Res Comm 149, 276-281 (1987); Kalebic et al., Cancer Res 54, 5531-5534 (1994); Adams et al., Cell Mol Life Sci 57, 1050-1063 (2000); Dricu et al., Glycobiology 9, 571-579 (1999); Kanter-Lewensohn et al., Melanoma Res 8, 389-397 (1998); Li et al., Cancer Immunol Immunothe. 49, 243-252 (2000), Arteaga et al., Breast Cancer Res Treatment 22, 101-106 (1992); Hailey et al., Mol Cancer Ther 1, 1349-1353 (2002), and many other publications.

In particular, the monoclonal antibody against IGF-1R called αIR-3 is widely used in the investigation of IGF-1R mediated processes and IGF-1R mediated diseases such as cancer. αIR-3 was described by Kull et al., J Biol Chem 258, 6561-6566 (1983). In the meantime, more than hundred publications have been published dealing with the investigation and therapeutic use of αIR-3 with regard to its antitumor effect, alone and together with cytostatic agents such as doxorubicin and vincristine. αIR-3 is a murine monoclonal antibody which is known to inhibit binding of IGF-1 but not IGF-2 to IGF-1R (Steele-Perkins and Roth, Biochem Biophys Res Comm 171, 1244-1251 (1990). At high concentrations αIR-3 stimulates tumor cell proliferation and IGF-1R phosphorylation (Bergmann et al., Cancer Res 55, 2007-2011 (1995); Kato et al., J Biol Chem 268, 2655-2661 (1993)). There exist other antibodies (e.g. 1H7, Li et al., Cancer Immunol Immunother 49, 243-252 (2000)) which inhibit IGF-2 binding to IGF-1R more potently than IGF-1 binding.

Particularly suitable anti-IGF-1R antibodies useful for the invention are chimeric, humanized or fully human antibodies. Such antibodies are e.g. the fully human IgG1 mAb cixutumumab/IMC-A12 (described in Burtrum et al., Cancer Res 63, 8912-21 (2003); Rowinsky et al., Clin Cancer Res 13, 5549s-5555s (2007), the fully human IgG1 mAb AMG-479 (described in Beltran et al., Mol Cancer Ther 8, 1095-1105 (2009); Tolcher et al., J Clin Oncol 27, 5800-7 (2009)), the humanized IgG1 mAb MK-0646/h7C10 (described in Goetsch et al., Int J Cancer 113, 316-28 (2005); Broussas et al., Int J Cancer 124, 2281-93 (2009); Hidalgo et al., J Clin Oncol 26, abstract 3520 (2008); Atzori et al., J Clin Oncol 26, abstract 3519 (2008)), the humanized IgG1 mAb AVE1642 (described in Descamps et al., Br J Cancer 100, 366-9 (2009); Tolcher et al., J Clin Oncol 26, abstract 3582 (2008); Moreau et al., Blood 110, abstract 1166 (2007); Maloney et al., Cancer Res 63, 5073-83 (2003)), the fully human IgG2 mAb figitumumab/CP-751,871 (Cohen et al., Clin Cancer Res 11, 2063-73 (2005); Haluska et al., Clin Cancer Res 13, 5834-40 (2007); Lacy et al., J Clin Oncol 26, 3196-203 (2008); Gualberto & Karp, Clin Lung Cancer 10, 273-80 (2009), the fully human IgG1 mAb SCH-717454 (described in WO 2008/076257 or Kolb et al., Pediatr Blood Cancer 50, 1190-7 (2008)), the 2.13.2. mAb (described in U.S. Pat. No. 7,037,498 (WO 2002/053596)) or the fully human IgG4 mAb BIIB022.

More examples of suitable human antibodies against IGF-1R are described in WO 2002/053596, WO 2004/071529, WO 2005/016967 WO 2006/008639, US 2005/0249730, US 2005/0084906, WO 2005/058967, WO 2006/013472, US 2003/0165502, WO 2005/082415, WO 2005/016970, WO 2003/106621, WO 2004/083248 and WO 2003/100008.

A summary of the state of the art of anti-IGF-1R antibodies and their properties and characteristics is given by Gualberto & Pollak, Oncogene 28, 3009-3021 (2009) or Atzori et al., Targ Oncol 4, 255-266 (2009).

Preferably, the anti-IGF-1R antibodies useful for the present invention are fully human antibodies, the technology for the production of which is well known in the art (e.g. van Dijk and van de Winkel, Curr Opin Pharmaco 5, 368-374 (2001)).

Preferred anti-IGF-1R antibodies useful for the invention are described in WO 2005/005635, the entire content of which is incorporated herein by reference, and inhibit the binding of insulin-like growth factor-1 (IGF-1) and insulin-like growth factor-2 (IGF-2) to insulin-like growth factor-1 receptor (IGF-1R). As described in WO 2005/005635, said anti-IGF-1R antibodies are characterized in that they
a) are of IgG1 isotype,
b) show a ratio of IC₅₀ values of inhibition of the binding of IGF-1 to IGF-1R to the inhibition of binding of IGF-2 to IGF-1R of 1:3 to 3:1,
c) inhibit for at least 80%, preferably at least 90%, at a concentration of 5 nM IGF-1R phosphorylation in a cellular phosphorylation assay using HT29 cells (a human colon carcinoma cell line known in the art) in a medium containing 0.5% heat inactivated fetal calf serum (FCS) when compared to such an assay without said antibody, and
d) show no IGF-1R stimulating activity (no signaling, no IGF-1 mimetic activity) measured as protein kinase B (PKB) phosphorylation at a concentration of 10 μM in a cellular phosphorylation assay using 3T3 cells providing 400,000 to 600,000 molecules IGF-1R per cell in a medium containing 0.5% heat inactivated FCS when compared to such an assay without said antibody.

The anti-IGF-1R antibodies useful for the invention are preferably monoclonal antibodies and, in addition, chimeric antibodies (human constant chain), humanized antibodies and especially preferably fully human antibodies. Preferred anti-IGF-1R antibodies useful for the invention bind to human IGF-1R (SwissProt P08069; EC 2.7.10.1) in competition to antibody 18, i.e. they bind to the same epitope of IGF-1R as antibody 18, which is described in WO 2005/005635. Preferred anti-IGF-1R antibodies are further characterized by an affinity to IGF-1R of 10⁻⁸ M (K_{D}) or less, preferably of about 10⁻⁹ to 10⁻¹³ M, and preferably show no detectable concentration-dependent inhibition of insulin binding to the insulin receptor.

Preferred anti-IGF-1R antibodies useful for the invention comprise complementarity determining regions (CDRs) having the following sequences:
a) an antibody heavy chain comprising as CDRs CDR1, CDR2 and CDR3 of SEQ ID NO:41 or 43;
b) an antibody light chain comprising as CDRs CDR1, CDR2 and CDR3 of SEQ ID NO:42 or 44.

Preferably, the anti-IGF-1R antibodies useful for the invention comprise an antibody heavy chain variable domain amino acid sequence of SEQ ID NO:41 and an antibody light chain variable domain amino acid sequence of SEQ ID NO:42, or an antibody heavy chain variable domain amino acid sequence of SEQ ID NO:43 and an antibody light chain variable domain amino acid sequence of SEQ ID NO:44.

The heavy and light chain amino acid sequences of preferred anti-IGF-1R antibodies useful for the invention are summarized in Table 4.

TABLE 4
Heavy and light chain variable domain
amino acid sequences of preferred anti-
IGF-1R antibodies.

| | | SEQ |
|---|---|---|
| | | ID |
| CONSTRUCT | AMINO ACID SEQUENCE | NO |
| V_{H} | QVELVESGGGVVQPGRSQRLSCAASGFTFSSY | 41 |
| | WGMHVRQAPGKGLEWVAIIWFDGSSTYYADSV | |
| | RGRFTISRDNSKNTLYLQMNSLRAEDTAVYFC | |
| | ARELGRRYFDLWGRGTLVSVSS | |
| V_{L} | EIVLTQSPATLSLSPGERATLSCRASQSVSSY | 42 |
| | LAWYQQKPGQAPRLLIYDASKRATGIPARFSG | |
| | SGSGTDFTLTISSLEPEDFAVYYCQQRSKWPP | |
| | WTFGQGTKVESK | |
| V_{H} | QVQLVESGGGVVQPGRSLRLSCAASGFTFSSY | 43 |
| | GMHWVRQAPGKGLEWMAIIWFDGSSKYYGDSV | |
| | KGRFTISRDNSKNTLYLQMNSLRADDTAVYYC | |
| | ARELGRRYFDLWGRGTLVTVSS | |
| V_{L} | EIVLTQSPATLSLSPGERATLSCRASQSVSSY | 44 |
| | LAWYQQKPGQAPRLLIYDASNRATGIPARFSG | |
| | SGSGTDFTLTISSLEPEDFAVYYCQQRSKWPP | |
| | WTFGQGTKVEIK | |

Preferred anti-IGF-1R antibodies useful for the invention are of IgG1 isotype and therefore provide C1q complement binding and induce complement-dependent cytotoxicity (CDC). Preferred antibodies are further characterized by the ability to bind Fcy receptors and to induce antibody-dependent cell-mediated cytotoxicity (ADCC). Preferred anti-IGF-1R antibodies inhibit the binding of IGF-1 and IGF-2 to IGF-1R in vitro and in vivo, preferably in about an equal manner for IGF-1 and IGF-2.

Preferred anti-IGF-1R antibodies useful for the invention are obtainable from the hybridoma cell lines <IGF-1R>HUMAB-Clone 18 and <IGF-1R>HUMAB-Clone 22, which are described in WO 2005/005635 and are deposited with Deutsche Sammlung von Mikroorganismen and Zellkulturen GmbH (DSMZ), Germany, under deposition numbers DSM ACC 2587 and DSM ACC 2594, respectively.

In another preferred embodiment, anti-IGF-1R antibodies useful for the invention have been glycoengineered to have an altered oligosaccharide structure in the Fc region.

Specifically, preferred anti-IGF-1R antibodies have an increased proportion of non-fucosylated oligosaccharides in the Fc region as compared to non-glycoengineered antibodies. Preferably, the percentage of non-fucosylated oligosaccharides is at least 20%, more preferably at least 50-70%, most preferably at least 75%. Anti-IGF-1R antibodies useful for the invention having such percentages of non-fucosylated oligosaccharides are further termed as partially fucosylated. The non-fucosylated oligosaccharides may be of the hybrid or complex type.

Preferred anti-IGF-1R antibodies may also have an increased proportion of bisected oligosaccharides in the Fc region. Preferably, the percentage of bisected oligosaccharides in the Fc region of the antibody is at least 50%, more preferably, at least 60%, at least 70%, at least 80%, or at least 90%, and most preferably at least 90-95% of the total oligosaccharides.

Particularly preferred anti-IGF-1R antibodies have an increased proportion of bisected, non-fucosylated oligosaccharides in the Fc region. The bisected, non-fucosylated oligosaccharides may be either hybrid or complex. Specifically, anti-IGF-1R antibodies are preferred in which at least 15%, more preferably at least 20%, more preferably at least 25%, more preferably at least 30%, more preferably at least 35% of the oligosaccharides in the Fc region of the antibody are bisected, non-fucosylated.

In another preferred embodiment, the anti-IGF-1R antibodies useful for the invention have been glycoengineered to have an altered oligosaccharide structure in the Fc region, as decribed in WO 2008/077546, the entire content of which is incorporated herein by reference.

Specifically, preferred anti-IGF-1R antibodies are glycosylated with a sugar chain at Asn 297, and are partially fucosylated, i.e. have an increased proportion of non-fucosylated oligosaccharides as compared to non-glycoengineered antibodies, as described hereinbefore.

Preferably the amount of N-glycolylneuraminic acid (NGNA) within the sugar chain at Asn 297 is 1% or less and/or the amount of N-terminal alpha-1,3-galactose is 1% or less.

Preferably the amount of NGNA within said sugar chain is 0.5% or less, more preferably 0.1% or less and even not detectable (by liquid chromatography-mass spectrometry (LCMS)).

Preferably the amount of N-terminal alpha-1,3-galactose within said sugar chain is 0.5% or less, more preferably 0.1% or less and even not detectable (LCMS).

According to the invention “amount of fucose/N-glycolylneuraminic acid/N-terminal alpha-1,3-galactose” means the amount of glycostructures attached to Asn 297 containing said sugar, relative to the sum of all glycostructures attached to Asn 297 (e. g. complex, hybrid and high mannose structures) measured by MALDI-TOF mass spectrometry and calculated as average value.

The sugar chains preferably show the characteristics of N-linked glycans attached to Asn 297 of an anti-IGF-1R antibody recombinantly expressed in a CHO cell.

Preferred anti-IGF-1R antibodies useful for the invention are glycosylated with a sugar chain at Asn 297, and are characterized in showing high binding affinity to the FcγRIIIα and/or increased effector function, such as increased Fc-mediated cellular cytotoxicity (including increased antibody-dependent cell-mediated cytotoxicity (ADCC)), increased antibody-dependent cellular phagocytosis (ADCP), increased cytokine secretion, increased immune complex-mediated antigen uptake by antigen-presenting cells, increased binding to natural killer (NK) cells, increased binding to macrophages, increased binding to monocytes, increased binding to polymorphonuclear cells, increased direct signaling inducing apoptosis, increased crosslinking of target-bound antibodies, increased dendritic cell maturation, or increased T cell priming.

The most preferred anti-IGF-1R antibody useful for the present invention is the human antibody obtainable from the hybridoma cell line <IGF-1R>HUMAB-Clone 18, which comprises the antibody heavy chain of SEQ ID NO:41 and the antibody light chain of SEQ ID NO:42. This antibody is termed “rhu anti-IGF-1R mAb 18”. rhu anti-IGF-1R mAb 18 may or may not be partially fucosylated, i.e. glycoengineered as described above, to have an increased proportion of non-fucosylated oligosaccharides in the Fc region as compared to non-glycoengineered antibodies.

Techniques for the production and isolation of monoclonal antibodies and antibody fragments, methods for humanizing non-human antibodies, as well as procedures for recombinant production and purification of antibodies are well-known in the art. A description of such techniques, including relevant references, is given e.g. in WO 2006/082515 and WO 2005/005635.

It is known that several mechanisms are involved in the therapeutic efficacy of antibodies against growth factor receptors such as EGFR or IGF-1R. These include blocking of ligand (e.g., EGF, TGF-α, IGF-1, IGF-2 etc.) binding to their receptors and subsequent activation of signaling pathways, antibody dependent cell-mediated cytotoxicity (ADCC), complement-dependent cytotoxicity (CDC) and the induction of growth arrest, apoptosis or terminal differentiation.

The therapeutic efficacy of the humanized IgG-class anti-EGFR antibody and the anti-IGF-1R antibody useful for the present invention can be enhanced by producing them in host cells that further express a polynucleotide encoding a polypeptide having β(1,4)-N-acteylglucosaminyltransferase (GnTIII) activity, as described in WO 99/54342, WO 2006/082515 and WO 2008/077546, which results in antibodies having a reduced proportion of fucosylated oligosaccharides in the Fc region (termed “partially fucosylated” antibodies). In a preferred aspect, the polypeptide having GnTIII activity is a fusion polypeptide comprising the catalytic domain of GnTIII and the Golgi localization domain of a heterologous Golgi resident polypeptide, such as the Golgi localization domain of mannosidase II, mannosidase I, β(1,2)-N-acteylglucosaminyltransferase I (GnTI), β(1,2)-N-acteylglucosaminyltransferase II (GnTII) or α1-6 core fucosyltransferase, preferably mannosidase II or GnTI. Methods for generating such fusion polypeptides and using them to produce antibodies with increased effector functions are disclosed in U.S. Provisional Patent Appl. No. 60/495,142, U.S. Patent Appl. Publ. No. 2004/0241817 A1 and WO 2004/065540, the entire contents of each of which are expressly incorporated herein by reference.

The partially fucosylated humanized IgG-class anti-EGFR antibody and the partially fucosylated anti-IGF-1R antibody may exhibit increased Fc receptor binding affinity and/or increased effector function as a result of the oligosaccharide modification. Preferably, the increased Fc receptor binding affinity is increased binding to a Fcγactivating receptor, such as the FcγRIIIα receptor. The increased effector function is preferably an increase in one or more of the following: increased Fc-mediated cellular cytotoxicity (including increased antibody-dependent cell-mediated cytotoxicity (ADCC)), increased antibody-dependent cellular phagocytosis (ADCP), increased cytokine secretion, increased immune-complex-mediated antigen uptake by antigen-presenting cells, increased binding to NK cells, increased binding to macrophages, increased binding to polymorphonuclear cells (PMNs), increased binding to monocytes, increased crosslinking of target-bound antibodies, increased direct signaling inducing apoptosis, increased dendritic cell maturation, and increased T cell priming.

Partially fucosylated antibodies can be produced in a host cell expressing a polynucleotide encoding the antibody and a polynucleotide encoding a polypeptide with GnTIII activity, or a vector comprising such polynucleotides. Production of the humanized IgG-class anti-EGFR antibody or the anti-IGF-1R antibody in said host cell comprises the following steps (a) culturing a host cell engineered to express at least one nucleic acid encoding a polypeptide having GnTIII activity under conditions which permit the production of the antibody, wherein said polypeptide having GnTIII activity is expressed in an amount sufficient to modify the oligosaccharides in the Fc region of said antibody produced by said host cell; and (b) isolating said antibody.

The humanized IgG-class anti-EGFR antibody and the anti-IGF-1R antibody may also exhibit increased Fc receptor binding affinity and/or increased effector function, preferably increased Fc-mediated cellular cytotoxicity, most preferably increased ADCC, as a result of one or more modification other than oligosaccharide modification. Such modifications include, but are not limited to, amino acid modifications in the Fc region (Fc region variants) as described below.

A variety of host cells and expression vector systems can be used for the production of antibodies and are well known in the art. Suitable host cells for expressing the humanized IgG-class EGFR antibody and the anti-IGF-1R antibody useful for the invention include cultured cells, e.g. cultured mammalian cells such as CHO cells, HEK293-EBNA cells, BHK cells, NS0 cells, SP2/0 cells, YO myeloma cells, P3×63 mouse myeloma cells, PER cells, PER.C6 cells or hybridoma cells, E. coli cells, yeast cells, insect cells, and plant cells, to name only a few, but also cells comprised within a transgenic animal, transgenic plant or cultured plant or animal tissue. Detailed information about the production of the IgG-class humanized anti-EGFR antibody and the anti-IGF-1R antibody can be found e.g. in WO 2006/082515, WO 2005/005635 and WO 2008/077546 and the references cited therein.

The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described for example in WO 2008/077546. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. The relative amount of fucose is the percentage of fucose-containing structures related to all glycostructures identified in an N-Glycosidase F treated sample (e. g. complex, hybrid and high mannose structures) by MALDI-TOF MS. Such fucosylation variants may have improved ADCC function.

In a first aspect, the present invention provides a humanized IgG-class anti-EGFR antibody and an anti-IGF-1R antibody, for combined use in treating cancer. In one embodiment said IgG-class anti-EGFR antibody comprises (a) in the heavy chain variable domain a CDR1 of SEQ ID NO:1, a CDR2 of SEQ ID NO:16 and a CDR3 of SEQ ID NO:31, and (b) in the light chain variable domain a CDR1 of SEQ ID NO:33, a CDR2 of SEQ ID NO:34 and a CDR3 of SEQ ID No:35. In another embodiment the humanized IgG-class anti-EGFR antibody comprises the heavy chain variable domain amino acid sequence of SEQ ID NO:38 and the light chain variable domain amino acid sequence of SEQ ID NO:39. In yet another embodiment, the humanized IgG-class anti-EGFR antibody comprises a human IgG1 Fc region of SEQ ID NO:40. In certain embodiments, the humanized IgG-class anti-EGFR antibody is glycoengineered to have an altered oligosaccharide structure in the Fc region. In a preferred embodiment the humanized IgG-class anti-EGFR antibody has an increased proportion of non-fucosylated oligosaccharides in its Fc-region compared to a non-glycoengineered antibody. In another preferred embodiment the humanized IgG-class anti-EGFR antibody has at least 20%, more preferably at least 50%, most preferably at least 75% non-fucosylated oligosaccharides in its Fc-region. In certain embodiments the humanized IgG-class anti-EGFR antibody has increased effector function, preferably increased Fc-mediated cellular cytotoxicity, most preferably increased ADCC, and/or increased Fc receptor binding compared to a non-glycoengineered antibody. In certain embodiments the humanized IgG-class anti-EGFR antibody is modified to have increased effector function, preferably increased Fc-mediated cellular cytotoxicity, most preferably increased ADCC, and/or increased Fc receptor binding compared to an unmodified antibody. In one embodiment said anti-IGF-1R antibody is a humanized or a human antibody, preferably a human antibody. In certain embodiments said anti-IGF-1R antibody is glycoengineered to have an altered oligosaccharide structure in the Fc region. In a preferred embodiment the anti-IGF-1R antibody has an increased proportion of non-fucosylated oligosaccharides in its Fc-region compared to a non-glycoengineered antibody. In another preferred embodiment the anti-IGF-1R antibody has at least 20%, more preferably at least 50%, most preferably at least 75% non-fucosylated oligosaccharides in its Fc-region. In certain embodiments, the anti-IGF-1R antibody has increased effector function, preferably increased Fc-mediated cellular cytotoxicity, most preferably increased ADCC, and/or increased Fc receptor binding compared to a non-glycoengineered antibody. In certain embodiments the anti-IGF-1R antibody is modified to have increased effector function, preferably increased Fc-mediated cellular cytotoxicity, most preferably increased ADCC, and/or increased Fc receptor binding compared to an unmodified antibody. In certain embodiments the anti-IGF-1R antibody binds to human IGF-1R in competition to antibody 18. In certain embodiments the anti-IGF-1R antibody comprises a heavy chain variable domain amino acid sequence of SEQ ID NO:41 or SEQ ID NO:43 and a light chain variable domain amino acid sequence of SEQ ID NO:42 or SEQ ID NO:44. In a specific embodiment the IGF-1R antibody comprises the heavy chain variable domain amino acid sequence of SEQ ID NO:41 and the light chain variable domain amino acid sequence of SEQ ID NO:42. In another specific embodiment, the IGF-1R antibody comprises the heavy chain variable domain amino acid sequence of SEQ ID NO:43 and the light chain variable domain amino acid sequence of SEQ ID NO:44. In yet another specific embodiment the anti-IGF-1R antibody is obtainable from the hybridoma cell line DSM ACC 2587 or the hybridoma cell line DSM ACC 2594. In certain embodiments the humanized IgG-class anti-EGFR antibody and the anti-IGF-1R antibody are both glycoengineered to have an altered oligosaccharide structure in the Fc region. In a preferred embodiment the humanized IgG-class anti-EGFR antibody and the anti-IGF-1R antibody both have an increased proportion of non-fucosylated oligosaccharides in their Fc-regions compared to the non-glycoengineered antibodies. In another preferred embodiment the humanized IgG-class anti-EGFR antibody and the anti-IGF-1R antibody both have an increased effector function, preferably increased Fc-mediated cellular cytotoxicity, most preferably increased ADCC, and/or increased Fc receptor binding compared to non-glycoengineered antibodies. In certain embodiments the antibodies are administered together. In other embodiments the antibodies are administered separately. In certain embodiments the antibodies are contained in the same formulation. In other embodiments the antibodies are contained in different formulations. In certain embodiments, the antibodies are administered simultaneously. In other embodiments the antibodies are administered sequentially. In certain embodiments the antibodies are administered by the same route, preferably administered parenterally, most preferably administered intravenously. In other embodiments the antibodies are administered by different routes, for example one antibody being administered parenterally and one antibody administered non-parenterally or the two antibodies being administered by two different routes of parenteral administration. In certain embodiments one or more additional therapeutic agents or treatments are used with the antibodies, for example one or more anti-cancer agents and/or radiation therapy.

In another aspect the invention provides a pharmaceutical composition, in particular for use in treating cancer, which comprises a humanized IgG-class anti-EGFR antibody and an anti-IGF-1R antibody as active ingredients, and a pharmaceutically acceptable carrier. The antibodies are as described hereinbefore and may incorporate any of the features, singly or in combination, described in the preceding paragraphs in relation to the antibodies useful for the invention. In certain embodiments, the composition additionally comprises one or more other therapeutically active ingredients or adjuvants. Other therapeutic agents may include cytotoxic, chemotherapeutic or anti-cancer agents, or agents which enhance the effects of such agents.

The data presented in the Examples herein below demonstrate that co-administration of an anti-IGF-1R antibody with a humanized IgG-class anti-EGFR antibody is effective for treatment of advanced cancers, such as Non Small Cell Lung Cancer (NSCLC). Accordingly, in one aspect the present invention provides a method for the treatment of cancer, comprising administering to a subject in need a humanized IgG-class anti-EGFR antibody and an anti-IGF-1R antibody. The antibodies are as described hereinbefore and may incorporate any of the features, singly or in combination, described in the preceding paragraphs in relation to the antibodies useful for the invention. In one embodiment a therapeutically effective amount of a combination of a humanized IgG-class anti-EGFR antibody and an anti-IGF-1R antibody is administered to a subject in need of such treatment. A therapeutically effective amount of a combination of a humanized IgG-class anti-EGFR antibody and an anti-IGF-1R antibody may be a therapeutically effective amount of each of the antibodies. Alternatively, in order to reduce the side effects caused by the treatment of cancer, a therapeutically effective amount of a combination of a humanized IgG-class anti-EGFR antibody and an anti-IGF-1R antibody may be amounts of the two antibodies that are effective to produce an additive, or a superadditive or synergistic antitumor effect, and that in combination are effective at inhibiting the growth of the tumor, but which may be sub-therapeutic amounts of one or both antibodies if they were used alone. In specific embodiments of the method for the treatment of cancer according to the invention, the humanized IgG-class anti-EGFR antibody and the anti-IGF-1R antibody are intended for administration to the patient together or separately, simultaneously or sequentially (in any order), in the same or in different formulations, by the same or different routes, and with or without additional agents or treatments, such as other anti-cancer drugs or radiation therapy.

In a further aspect, the present invention further provides a method for manufacturing a medicament for the treatment of cancer, characterized in that a therapeutically effective amount of a combination of a humanized IgG-class anti-EGFR antibody and an anti-IGF-1R antibody is used. The humanized IgG-class anti-EGFR antibody and the anti-IGF-1R antibody are as described hereinbefore and may incorporate any of the features, singly or in combination, described in the preceding paragraphs in relation to the antibodies useful for the invention. As described above, a therapeutically effective amount of a combination of a humanized IgG-class anti-EGFR antibody and an anti-IGF-1R antibody may be a therapeutically effective amout of each of the antibodies, or amounts of the two antibodies that are effective to produce an additive, or a superadditive or synergistic antitumor effect, and that in combination are effective at inhibiting the growth of the tumor, but which may be sub-therapeutic amounts of one or both antibodies if they were used alone. In specific embodiments according to the invention, the two antibodies are intended for administration to the patient together or separately, simultaneously or sequentially, in the same or in different formulations, by the same or different routes, and with or without additional agents or treatments.

In one aspect, the present invention provides a kit, useful for the treatment of cancer, comprising a single container comprising both the humanized IgG-class anti-EGFR antibody and the anti-IGF-1R antibody. In another aspect, the present invention provides a kit comprising a first container comprising the humanized IgG-class anti-EGFR antibody and a second container comprising the anti-IGF-1R antibody. The antibodies are as described hereinbefore and may incorporate any of the features, singly or in combination, described in the preceding paragraphs in relation to the antibodies useful for the invention. In a preferred embodiment, the kit containers further include a pharmaceutically acceptable carrier. In some embodiments the kit further includes a sterile diluent, which is preferably stored in a separate additional container. In certain embodiments the kit further includes a package insert comprising printed instructions directing the use of the combined treatment as a method for treating cancer.

The present invention is intended for the treatment of cancer. Accordingly the subject in need is a human, horse, swine, bovine, mouse, rat, dog, cat, bird or other warm-blooded animal, preferably a human, in need of treatment of cancer or a precancerous condition or lesion. The cancer is preferably any cancer treatable, either partially or completely, by administration of a combination of a humanized IgG-class anti-EGFR antibody and an anti-IGF-1R antibody as described hereinbefore, i.e. a disorder that relates to EGFR and/or IGF-1R expression, in particular, a cell proliferation disorder wherein EGFR and/or IGF-1R is expressed, and more particularly, wherein EGFR and/or IGF-1R is abnormally expressed (e.g. overexpressed). The cancer may be, for example, lung cancer, non small cell lung cancer (NSCLC), bronchioloalveolar carcinoma, bone cancer, pancreatic cancer, skin cancer, cancer of the head or neck, squameous cell carcinoma, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, including refractory versions of any of the above cancers, or a combination of one or more of the above cancers. Also included are cancer metastases. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions. Preferably, the cancer is lung cancer and most preferably non-small cell lung cancer (NSCLC).

In some aspects of this invention, the humanized IgG-class anti-EGFR antibody and the anti-IGF-1R antibody as described hereinbefore may be administered in combination with one or more anti-cancer agents, wherein said anti-cancer agents may be selected from the groups of microtubule disruptors (e.g. vinca alkaloids such as vinblastine or vincristine, taxanes such as docetaxel or paclitaxel, epothilones such as ixabepilone), antimetabolites (e.g. anti-folates such as methotrexate or aminopterin, anti-purines such as fludarabine, 6-mercaptopurine or 6-thioguanine, anti-pyrimidines such as 5-fluorouracil, capecitabine or gemcitabine, hydroxyurea), topoisomerase inhibitors (e.g. camptothecin, irinotecan or podophyllotoxins such as etoposide), DNA intercalators (e.g. doxorubicin, daunorubicin, actinomycin, bleomycin), alkylating agents (e.g. cyclophosphamide, chlorambucil, nitrosureas such as carmustine or nimustine, streptozocin, busulfan, cisplatin, oxaliplatin, triethylenemelamine, dacarbazine), hormonal therapies (e.g. glucocorticoids, aromatase inhibitors such as tamoxifene, antiandrogens such as flutamide, gonadotropin-releasing hormone (GnRH) analogs such as leuprolide), antibiotics, kinase inhibitors (e.g. erlotinib, gefitinib, imatinib), receptor antagonists, enzyme inhibitors (e.g. cyclin-dependent kinase (CDK) inhibitors), amino acid-depleting enzymes (e.g. asparaginase), leucovorin, retinoids, activators of tumor cell apoptosis, and antiangiogenic agents.

The humanized IgG-class anti-EGFR antibody and/or the anti-IGF-1R antibody useful for the invention may also be conjugated to a cytotoxic agent such as a chemotherapeutic agent, a toxin (e.g. an enzymatically active toxin of bacterial, fungal, plant or animal origin, or fragments thereof), a radioactive isotope, or to a prodrug of a cytotoxic agent.

The humanized IgG-class anti-EGFR antibody and the anti-IGF-1R antibody as used in the invention, or the pharmaceutical composition according to the invention can be administered in any effective manner known in the art, such as by oral, topical, intravenous, intraperitoneal, intralymphatic, intramuscular, intra-articular, subcutaneous, intranasal, intra-ocular, vaginal, rectal, or intradermal routes, or by injection directly into the tumor. The choice of a route of administration depends upon the type of cancer being treated and the medical judgement of the prescribing physician as based, e.g., on the results of published clinical studies. The two antibodies can be administered by the same or by different routes. Preferably, the humanized IgG-class anti-EGFR antibody and the anti-IGF-1R antibody as used in the invention, or the pharmaceutical composition according to the invention are administered parenterally, most preferably intravenously. The antibodies or the composition can be administered by controlled release means and/or delivery devices.

The combination of a humanized IgG-class anti-EGFR antibody and an anti-IGF-1R antibody as used in the present invention should be administered in a therapeutically effective amount, meaning that each of the antibodies is given in therapeutically effective dose, or that the amounts of the two antibodies are effective to produce an additive, or a superadditive or synergistic antitumor effect, so that in combination they are effective at inhibiting the growth of the tumor, although they would be sub-therapeutic amounts if the antibodies were used alone.

The most effective mode of administration and dosage regimen for the humanized IgG-class anti-EGFR antibody and the anti-IGF-1R antibody as used in the invention or the pharmaceutical compositions according to the invention depends on a variety of factors, including the severity and course of the disease, the patient's general health, age, body weight, sex, diet and response to treatment, the time and route of administration, the rate of excretion, combinations with other drugs, and the judgment of the treating physician. Accordingly, the dosages of the antibodies or compositions should be titrated to the individual patient. Nevertheless, an effective dose of the antibodies or compositions of this invention will generally be in the range of from about 0.01 to about 2000 mg/kg. Typically, the therapeutically effective amount of the antibody administered parenterally per dose will be in the range of about 1 to 25 mg/kg of patient body weight per day. In one aspect, the effective dose is in the range of from about 1.0 mg/kg to about 25.0 mg/kg. In a more specific aspect, the dose is in the range of from about 1.5 mg/kg to about 15 mg/kg. In other aspects, the dose is in the range of from about 1.5 mg/kg to about 4.5 mg/kg, or from about 4.5 mg/kg to about 15 mg/kg. The dose of the present invention may also be any dose within these ranges, including, but not limited to, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, 10.0 mg/kg, 10.5 mg/kg, 11.0 mg/kg, 11.5 mg/kg, 12.0 mg/kg, 12.5 mg/kg, 13.0 mg/kg, 13.5 mg/kg, 14.0 mg/kg, 14.5 mg/kg, or 15.0 mg/kg.

As noted above, however, these suggested amounts of humanized IgG-class anti-EGFR antibody and of anti-IGF-1R antibody are subject to a great deal of therapeutic discretion. The key factor in selecting an appropriate dose and scheduling is the result obtained, as indicated above. For example, relatively higher doses may be needed initially for the treatment of ongoing and acute diseases. To obtain the most efficacious results, depending on the disease or disorder, the antagonist is administered as close to the first sign, diagnosis, appearance, or occurrence of the disease or disorder as possible or during remissions of the disease or disorder.

In the case of anti-EGFR antibodies used to treat tumors, optimum therapeutic results have generally been achieved with a dose that is sufficient to completely saturate the EGF receptors on the target cells. The dose necessary to achieve saturation will depend on the number of EGF receptors expressed per tumor cell (which can vary significantly between different tumor types). Serum concentrations as low as 30 nM have been effective in treating some tumors, while concentrations above 100 nM may be necessary to achieve optimum therapeutic effect with other tumors. The dose necessary to achieve saturation for a given tumor can be readily determined in vitro by radioimmunoassay or immunoprecipitation. Similar considerations can be made for determining the dose of anti-IGF-1R antibodies.

The dosages of the present invention may, in some cases, be determined by the use of predictive biomarkers. Predictive biomarkers are molecular markers that are used to determine (i.e., observe and/or quanitate) a pattern of expression and/or activation of tumor-related genes or proteins, or cellular components of a tumor-related signalling pathway. Elucidating the biological effects of targeted therapies in tumor tissue and correlating these effects with clinical response helps identify the predominant growth and survival pathways operative in tumors, thereby establishing a profile of likely responders and conversely providing a rationale for designing strategies to overcome resistance. For example, biomarkers for anti-EGFR therapy may comprise molecules that are in the EGFR downstream signalling pathway that leads to a cell proliferation disorder including, but not limited to, Akt, RAS, RAF, MAPK, ERK1, ERK2, PKC, STAT3, STAT5 (Mitchell, Nat Biotech 22, 363-364 (2004); Becker, Nat Biotech 22; 15-18 (2004); Tsao and Herbst, Signal 4, 4-9 (2003)). Biomarkers for anti-EGFR therapy may also comprise growth factor receptors such as EGFR, ErbB-2 (HER2/neu), and ErbB-3 (HER3), and may be positive or negative predictors of patient response to anti-EGFR therapy. For example, the growth factor receptor ErbB-3 (HER3) was determined to be a negative predictive biomarker for the anti-EGFR antibody ABX-EGF (U.S. Patent Appl. Pub. No. 2004/0132097 A1).

Similarly, predictive biomarkers may be exploited to identify tumors responsive or resistant to anti-IGF-1R therapy.

Predictive biomarkers may be measured by assays that are well known in the art including, but not limited to detection and/or quantification of RNA by real-time reverse transcription PCR or microarray-based transcriptional profiling, detection and/or quantification of protein by immunohistochemistry, flow cytometry, immunofluorescence, capture-and-detection assays, Western blot, ELISA, reversed phase assays, and/or assays set forth in U.S. Patent Appl. Pub. No. 2004/0132097 A1, the entire contents of which are herein incorporated by reference. Predictive biomarkers for anti-EGFR therapy can be identified according to the techniques set forth in U.S. Patent Appl. Pub. No. 2003/0190689A1, the entire contents of which are hereby incorporated by reference.

In one aspect, the present invention provides a method for treating an EGFR-related disorder comprising predicting a response to anti-EGFR therapy in a human subject in need of treatment by assaying a sample from the human subject prior to therapy with one or a plurality of reagents that detect expression and/or activiation of predictive biomarkers for an EGFR-related disorder such as cancer; determining a pattern of expression and/or activation of one or more of the predictive biomarkers, wherein the pattern predicts the human subject's response to the anti-EGFR therapy; and administering to a human subject who is predicted to respond positively to anti-EGFR treatment a therapeutically effective amount of a composition comprising the humanized IgG-class anti-EGFR antibody. As used herein, “a human subject who is predicted to respond positively to anti-EGFR treatment” is one for whom anti-EGFR will have a measurable effect on the EGFR-related disorder (e.g., tumor regression/shrinkage) and for whom the benefits of anti-EGFR therapy are not outweighed by adverse effects (e.g. toxicity). As used herein, a sample means any biological sample from an organism, particularly a human, comprising one or more cells, including single cells of any origin, tissue or biopsy samples which has been removed from organs such as breast, lung, gastrointestinal tract, skin, cervix, ovary, prostate, kidney, brain, head and neck, or any other organ or tissue of the body, and other body samples including, but not limited to, smears, sputum, secretions, cerebrospinal fluid, bile, blood, lymph fluid, urine and feces.

For purposes of the present invention, “co-administration of”, “co-administering” and “combining” of a humanized IgG-class anti-EGFR antibody and an anti-IGF-1R antibody refer to any administration of the two antibodies, either separately or together, where the two antibodies are administered as part of an appropriate dose regimen designed to obtain the benefit of the combination therapy. Thus, the two active agents can be administered either as part of the same pharmaceutical composition or in separate pharmaceutical compositions. The anti-IGF-1R antibody can be administered prior to, at the same time as, or subsequent to the administration of the humanized IgG-class anti-EGFR antibody, or in some combination thereof Where the humanized IgG-class anti-EGFR antibody is administered to the patient at repeated intervals, e.g., during a standard course of treatment, the anti-IGF-1R antibody can be administered prior to, at the same time as, or subsequent to, each administration of the humanized IgG-class anti-EGFR antibody or some combination thereof, or at different intervals in relation to the humanized IgG-class anti-EGFR antibody treatment, or in a single dose prior to, at any time during, or subsequent to the course of treatment with the humanized IgG-class anti-EGFR antibody.

The humanized IgG-class anti-EGFR antibody will typically be administered to the patient in a dose regimen that provides for the most effective treatment of the cancer (from both efficacy and safety perspectives) for which the patient is being treated, as known in the art, and as disclosed, e.g. in WO 2006/082515.

As discussed above, the amount of the humanized IgG-class anti-EGFR antibody administered and the timing of the antibody administration will depend on the type (species, gender, age, weight, etc.) and condition of the patient being treated, the severity of the disease or condition being treated, and on the route of administration. For example, the humanized IgG-class anti-EGFR antibody can be administered to a patient in doses ranging from 0.1 to 100 mg/kg of body weight per day or per week in single or divided doses, or by continuous infusion. In some instances, dosage levels below the lower limit of the aforesaid range may be adequate, while in other cases still larger doses may be employed without causing any harmful side effect, provided that such larger doses are first divided into several small doses for administration throughout the day.

The humanized IgG-class anti-EGFR antibody and the anti-IGF-1R antibody as used according to the invention can be administered either separately or together by the same or different routes, and in a wide variety of different dosage forms.

Both the humanized IgG-class anti-EGFR antibody and the anti-IGF-1R antibody as used in the invention, as well as the pharmaceutical compositions according to the invention, may be in a variety of dosage forms which include, but are not limited to, liquid solutions or suspensions, emulsions, tablets, pills, dragées, powders, ointments, creams, suppositories or implants. The humanized IgG-class anti-EGFR antibody and/or the anti-IGF-1R antibody as used in the invention or the compositions according to the invention may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Mack Pub. Co. (1980).The preferred dosage form depends upon the mode of administration and the therapeutic application. Typically, the humanized IgG-class anti-EGFR antibody and/or the anti-IGF-1R antibody as used in the invention or the compositions according to the invention will be administered in injectable or infusible solutions. Injectable or infusible preparations must be sterile, which is readily accomplished by filtration through sterile filtration membranes.

Sustained-release preparations may be prepared, such as membrane-controlled sustained release systems, or polymer-based matrix systems. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The antibodies as used in the invention or pharmaceutical compositions according to the invention may be provided as bulk or conveniently presented in unit dosage forms, prepared by any of the methods well known in the art of pharmacy.

The humanized IgG-class anti-EGFR antibody and the anti-IGF-1R antibody as used in the invention, as well as the pharmaceutical composition according to the invention, will be formulated, dosed, and administered in a fashion consistent with good medical practice.

The optimal formulation of the humanized IgG-class anti-EGFR antibody and the anti-IGF-1R antibody as used in the invention, as well as the pharmaceutical composition according to the invention will depend on the particular disease or disorder being treated, the particular mammal being treated, the clinical condition of the individual patient, the cause of the disease or disorder, the site of delivery of the agent, the route of administration (e.g. parenteral, oral, topical, rectal), the scheduling of the administration, and other factors known to medical practitioners.

All formulations should be selected so as to avoid denaturation and/or degradation and loss of biological activity of the humanized IgG-class anti-EGFR antibody and/or the anti-IGF-1R antibody.

In practice, the humanized IgG-class anti-EGFR antibody and/or the anti-IGF-1R antibody can be combined as the active ingredient in intimate admixture with a pharmaceutical carrier according to conventional pharmaceutical compounding techniques. The carrier may take a wide variety of forms depending on the type of preparation desired for administration, e.g. parenteral (including intravenous). The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In addition to the carrier ingredients, the pharmaceutical formulations may also contain, as appropriate, other ingredients such as buffers, diluents, stabilizers, antioxidants, agents to render the formulation isotonic, flavoring agents, binders, surface-active agents, thickeners, lubricants, preservatives, wetting agents, emulsifying agents, dispersing agents, agents to disintegrate tablets and the like. The formulations may be prepared by any of the methods of pharmacy.

Pharmaceutical formulations of the present invention suitable for injection include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The formulations must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

For parenteral administration of either or both of the antibodies, solutions in sesame or peanut oil or in aqueous propylene glycol may be employed, as well as sterile aqueous solutions comprising the active agent or a corresponding water-soluble salt thereof. Such sterile aqueous solutions are preferably suitably buffered, e.g. with histidine, acetate or phosphate buffers, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

Therapeutic formulations containing the humanized IgG-class anti-EGFR antibody and/or the anti-IGF-1R antibody are prepared by mixing the antibody having the desired degree of purity with optional pharmaceutically acceptable carriers, excipients or stabilizers (Remington's Pharmaceutical Sciences, 16th edition, Mack Pub. Co. (1980)). They may be stored in the form of lyophilized formulations or aqueous solutions. Acceptable carriers, excipients, or stabilizers are non-toxic to recipients at the dosages and concentrations employed, and include buffers such as phosphate, citrate, histidine, acetate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone or polyethylene glycol (PEG); amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyoxyethylen-sorbitan fatty acid esters (Tween™) or polyoxyethylene-polyoxypropylene copolymers (Pluronic™)

Lyophilized formulations adapted for subcutaneous administration are described in WO 97/04801. Such lyophilized formulations may be reconstituted with a suitable diluent to a high protein concentration and the reconstituted formulation may be administered subcutaneously to the mammal to be treated herein.

Methods of preparing pharmaceutical compositions comprising antibodies or antigen binding fragments thereof are known in the art, and are described, e.g. WO 2006/082515. In view of the teaching of the present invention, methods of preparing pharmaceutical compositions comprising both the humanized IgG-class anti-EGFR antibody and the anti-IGF-1R antibody will be apparent from the above-cited publications and from other known references, such as Remington's Pharmaceutical Sciences, 18th edition, Mack Pub. Co. (1990). The combination compositions may be prepared by any of the methods of pharmacy.

The examples below explain the invention in more detail. The following preparations and examples are given to enable those skilled in the art to more clearly understand and to practice the present invention. The present invention, however, is not limited in scope by the exemplified aspects, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

Terms are used herein as generally used in the art, unless otherwise defined as follows.

As used herein, the term “antibody” is intended to include whole antibody molecules, including monoclonal, polyclonal and multispecific (e.g., bispecific) antibodies, as well as antibody fragments having the Fc region and retaining binding specificity, and fusion proteins that include a region equivalent to the Fc region of an immunoglobulin and that retain binding specificity. Also encompassed are antibody fragments that retain binding specificity including, but not limited to, V_{H} fragments, V_{L} fragments, Fab fragments, F(ab′)₂ fragments, scFv fragments, Fv fragments, minibodies, diabodies, triabodies, and tetrabodies (see e.g. Hudson and Souriau, Nat Med 9, 129-134 (2003)). Also encompassed are genetically engineered, recombinant, humanized, primatized and chimeric antibodies, as well as antibodies from different species such as mouse or human.

As used herein, the terms “monoclonal antibody” or “monoclonal antibody composition” as used herein refer to a preparation of antibody molecules of a single amino acid composition. Accordingly, the term “human monoclonal antibody” refers to antibodies displaying a single binding specificity which have variable and constant domains derived from human germline immunoglobulin sequences. In one embodiment, the human monoclonal antibodies are produced by a hybridoma which includes a B cell obtained from a transgenic non-human animal, e.g. a transgenic mouse, having a genome comprising a human heavy chain transgene and a human light chain transgene, fused to an immortalized cell.

As used herein, the term “chimeric antibody” refers to a monoclonal antibody comprising a variable region, i.e., binding region, from one source or species and at least a portion of a constant region derived from a different source or species, usually prepared by recombinant DNA techniques. Chimeric antibodies comprising a murine variable region and a human constant region are especially preferred. Such murine/human chimeric antibodies are the product of expressed immunoglobulin genes comprising DNA segments encoding murine immunoglobulin variable regions and DNA segments encoding human immunoglobulin constant regions. Other forms of “chimeric antibodies” encompassed by the present invention are those in which the class or subclass has been modified or changed from that of the original antibody. Such “chimeric” antibodies are also referred to as “class-switched antibodies”. Methods for producing chimeric antibodies involve conventional recombinant DNA and gene transfection techniques now well known in the art (see e.g. Morrison et al., Proc Natl Acad Sci USA 81, 6851-6855 (1984); U.S. Pat. Nos. 5,202,238 and 5,204,244).

As used herein, the term “humanized” is used to refer to an antigen-binding molecule derived from a non-human antigen-binding molecule, for example, a murine antibody, that retains or substantially retains the antigen-binding properties of the parent molecule but which is less immunogenic in humans, e.g. chimeric antibodies. Reduction of immunogenicity may be achieved by various methods including (a) grafting the entire non-human variable domains onto human constant regions to generate chimeric antibodies, (b) grafting only the non-human CDRs onto human framework and constant regions with or without retention of critical framework residues (e.g., those that are important for retaining good antigen binding affinity or antibody functions), (c) grafting only the non-human specificity-determining regions (SDRs; the residues critical for the antibody-antigen interaction) onto human framework and constant regions, or (d) transplanting the entire non-human variable domains, but “cloaking” them with a human-like section by replacement of surface residues. Such methods are disclosed in Morrison et al., Proc Natl Acad Sci USA 81, 6851-6855 (1984); Morrison and 0i, Adv Immunol 44, 65-92 (1988); Verhoeyen et al., Science 239, 1534-1536 (1988); Padlan, Molec Immun, 28, 489-498 (1991); Padlan, Molec Immun 31(3), 169-217 (1994), Kashmiri et al., Methods 36, 25-34 (2005), all of which are incorporated by reference in their entirety herein. There are generally three complementarity determining regions, or CDRs, (CDR1, CDR2 and CDR3) in each of the heavy and light chain variable domains of an antibody, which are flanked by four framework subregions (i.e. FR1, FR2, FR3, and FR4) in each of the heavy and light chain variable domains of an antibody: FR1-CDR1-FR2-CDR2-FR3-CDR3-FR4. A discussion of humanized antibodies can be found, inter alia, in U.S. Pat. No. 6,632,927, and in published U.S. Application No. 2003/0175269, both of which are incorporated herein by reference in their entirety.

Similarly, as used herein, the term “primatized” refers to an antibody derived from a non-primate antibody, e.g. a murine antibody, that retains or substantially retains the antigen-binding properties of the parent molecule, but which is less immunogenic in primates.

As used herein, the term “human antibody” or “fully human antibody” is intended to include antibodies having variable and constant regions derived from human germline immunoglobulin sequences. Human antibodies can be produced using various techniques known in the art. Human antibodies are described generally in van Dijk and van de Winkel, Curr Opin Pharmacol 5: 368-74 (2001) and Lonberg, Curr Opin Immunol 20:450-459 (2008). Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat Biotech 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMMOUSE® technology. Human variable regions from intact antibodies generated by such animals may be further modified, e.g. by combining with a different human constant region. Human antibodies can also be made by hybridoma-based methods. Human myeloma and mouse-human heteromyeloma cell lines for the production of human monoclonal antibodies have been described. See, e.g., Kozbor, J Immunol 133: 3001 (1984); Brodeur et al., Monoclonal Antibody Production Techniques and Applications, pp. 51-63 (Marcel Dekker, Inc., New York, 1987); and Boerner et al., J Immunol 147: 86 (1991). Human antibodies generated via human B-cell hybridoma technology are also described in Li et al., Proc Natl Acad Sci USA, 103:3557-3562 (2006). Additional methods include those described, for example, in U.S. Patent No. 7,189,826 (describing production of monoclonal human IgM antibodies from hybridoma cell lines) and Ni, Xiandai Mianyixue 26(4):265-268 (2006) (describing human-human hybridomas). Human hybridoma technology (Trioma technology) is also described in Vollmers and Brandlein, Histology and Histopathology 20(3):927-937 (2005) and Vollmers and Brandlein, Methods and Findings in Experimental and Clinical Pharmacology 27(3):185-91 (2005). Human antibodies may also be generated by isolating Fv or Fab clone variable domain sequences selected from human-derived phage display libraries. A variety of methods are known in the art for generating phage display libraries and screening such libraries for antibodies possessing the desired binding characteristics. Such methods are reviewed, e.g., in Hoogenboom et al., in Methods in Molecular Biology 178:1-37 (O'Brien et al., ed., Human Press, Totowa, NJ, 2001) and further described, e.g., in McCafferty et al., Nature 348: 552-554 (1990); Clackson et al., Nature 352: 624-628 (1991); Marks et al., J Mol Biol 222: 581-597 (1992); Marks and Bradbury, in Methods in Molecular Biology 248: 161-175 (Lo, ed., Human Press, Totowa, NJ, 2003); Sidhu et al., J Mol Biol 338(2): 299-310 (2004); Lee et al., J Mol Biol 340(5): 1073-1093 (2004); Fellouse, Proc Natl Acad Sci USA 101(34): 12467-12472 (2004); and Lee et al., J Immunol Methods 284(1-2): 119-132(2004). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Such variable domain sequences may then be combined with a desired human constant domain.

The “variable region” or “variable domain” (variable region of a light chain (V_{L}), variable region of a heavy chain (V_{H})) as used herein denotes each of the pair of light and heavy chains which is involved directly in binding the antibody to the antigen. The human light and heavy chain variable domains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain CDR3 regions play a particularly important role in the binding specificity/affinity of the antibodies useful for the invention and therefore provide a further object of the invention.

The terms “hypervariable region” or “antigen-binding portion of an antibody” when used herein refer to the amino acid residues of an antibody which are responsible for antigen-binding. The hypervariable region comprises amino acid residues of the “complementarity determining regions” or “CDRs”. “Framework” or “FR” regions are those variable domain regions other than the hypervariable region residues as herein defined. Therefore, the variable regions of the light and heavy chains of an antibody comprise from N- to C-terminus the domains FR1, CDR1, FR2, CDR2, FR3, CDR3, and FR4. Notably, CDR3 of the heavy chain is the region which contributes most to antigen binding. CDR and FR regions can be determined according to the standard definition of Kabat et al., “Sequences of Proteins of Immunological Interest”, 5th Ed. Public Health Service, National Institutes of Health, Bethesda, Md. (1991)) and/or those residues from a “hypervariable loop”.

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen binding sites found within the variable region of both heavy and light chain polypeptides. This particular region has been described by Kabat et al., “Sequences of Proteins of Immunological Interest”, National Institutes of Health, Bethesda (1983) and by Chothia et al., J Mol Biol 196, 901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 5 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 5
CDR Definitions¹

| | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table 5 is according to the numbering conventions set forth by Kabat et al. (see below).
²“AbM” refers to the CDRs as defined by Oxford Molecular's AbM antibody modeling software.

Kabat et al. also defined a numbering system for variable domain sequences that is applicable to any antibody. One of ordinary skill in the art can unambigously assign this system of “Kabat numbering” to any variable domain sequence, without reliance on any experimental data beyond the sequence itself. As used herein, “Kabat numbering” refers to the numbering system set forth by Kabat et al., “Sequences of Proteins of Immunological Interest”, National Institutes of Health, Bethesda (1983). Unless otherwise specified, references to the numbering of specific amino acid residue positions in an antigen binding molecule are according to the Kabat numbering system.

The “constant domains” are the parts of an antibody molecule other than the variable regions. They are not involved directly in binding the antibody to an antigen but are involved in the effector functions (e.g. ADCC, CDC). The constant domain of the antibodies useful for the invention is preferably of the IgG1 isotype. Human constant domains having these characteristics are described in detail by Kabat et al., “Sequences of Proteins of Immunological Interest”, National Institutes of Health, Bethesda (1991), and by Brüggemann et al., J Exp Med 166, 1351-1361 (1987); Love et al., Methods Enzymol 178, 515-527 (1989). The constant domains useful in the invention provide complement binding and Fc receptor binding. ADCC and optionally CDC are provided by the combination of variable and constant domains.

As used herein, the term “Fc region” is intended to refer to a C-terminal region of an IgG heavy chain. Although the boundaries of the Fc region of an IgG heavy chain might vary slightly, the human IgG heavy chain Fc region is usually defined to stretch from the amino acid residue at position Cys 226 to the carboxyl-terminus.

As used herein, the term “region equivalent to the Fc region of an immunoglobulin” is intended to include naturally occurring allelic variants of the Fc region of an immunoglobulin, as well as variants having alterations which produce substitutions, additions, or deletions but which do not decrease substantially the ability of the immunoglobulin to mediate effector functions (such as antibody dependent cell-mediated cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see e.g. Bowie et al., Science 247, 1306-1310 (1990).

Constant domains of human IgG1, IgG2 or IgG3 isotype are glycosylated at Asn 297. “Asn 297” according to the invention means amino acid asparagine located at about position 297 in the Fc region. Based on minor sequence variations of antibodies, Asn 297 can also be located some amino acids (usually not more than ±3 amino acids) upstream or downstream. For example, in one antibody useful for the invention (rhu anti-IGF-1R mAb 18) “Asn 297” is located at amino acid position 298.

As used herein, “a polypeptide having GnTIII activity” refers to polypeptides that are able to catalyze the addition of a N-acetylglucosamine (G1cNAc) residue in β-1-4 linkage to the β-linked mannoside of the trimannosyl core of N-linked oligosaccharides. This includes fusion polypeptides exhibiting enzymatic activity similar to, but not necessarily identical to, an activity of β(1,4)-N-acetylglucosaminyltransferase III, also known as β-1,4-mannosyl-glycoprotein 4-β-N-acetylglucosaminyl-transferase (EC 2.4.1.144), according to the Nomenclature Committee of the International Union of Biochemistry and Molecular Biology (NC-IUBMB), as measured in a particular biological assay, with or without dose dependency. In the case where dose dependency does exist, it need not be identical to that of GnTIII, but rather substantially similar to the dose-dependence in a given activity as compared to the GnTIII (i.e., the candidate polypeptide will exhibit greater activity or not more than about 25-fold less and, preferably, not more than about tenfold less activity, and most preferably, not more than about threefold less activity relative to the GnTIII).

As used herein, the term “Golgi localization domain” refers to the amino acid sequence of a Golgi-resident polypeptide which is responsible for anchoring the polypeptide in location within the Golgi complex. Generally, localization domains comprise amino terminal “tails” of an enzyme.

As used herein, the term “host cell” covers any kind of cellular system which can be engineered to generate the antibodies of the present invention. In one embodiment, the host cell is engineered to allow the production of an antibody with modified glycoforms. Preferably, the host cells have been engineered to express increased levels of one or more polypeptides having GnTIII activity. Host cells include cultured cells, e.g. cultured mammalian cells such as CHO cells, HEK293-EBNA cells, BHK cells, NSO cells, SP2/0 cells, YO myeloma cells, P3×63 mouse myeloma cells, PER cells, PER.C6 cells or hybridoma cells, E. coli cells, yeast cells, insect cells, and plant cells, to name only a few, but also cells comprised within a transgenic animal, transgenic plant or cultured plant or animal tissue.

As used herein, the term “effector function” refers to those biological activities attributable to the Fc region (a native sequence Fc region or amino acid sequence variant Fc region) of an antibody. Examples of antibody effector functions include, but are not limited to, Fc receptor binding affinity, antibody-dependent cell-mediated cytotoxicity (ADCC), antibody-dependent cellular phagocytosis (ADCP), cytokine secretion, immune complex-mediated antigen uptake by antigen-presenting cells, down-regulation of cell surface receptors, etc.

The terms “engineer”, “engineered”, “engineering”, “glycosylation engineering”, “glycoengineered”, as used herein, includes any manipulation of the glycosylation pattern of a naturally occurring or recombinant protein, polypeptide or a fragment thereof. Glycoengineering includes metabolic engineering of the glycosylation machinery of a cell, including genetic manipulations of the oligosaccharide synthesis pathways to achieve altered glycosylation of glycoproteins expressed in these cells. Furthermore, glycoengineering includes the effects of mutations and cell environment on glycosylation. In particular, glycoengineering can result in altered glycosyltransferase activity, such as altered glucosaminyltransferase and/or fucosyltransferase activity. The glycoengineering methodology that can be used with the antibodies useful for the present invention has been described in greater detail in U.S. Pat. No. 6,602,684, U.S. Pat. Appl. Publ. No. 2004/0241817 A1, U.S. Pat. Appl. Publ. No. 2003/0175884 Al, Provisional U.S. Patent Application No. 60/441,307, WO 99/54342 and WO 2004/065540, the entire contents of each of which are incorporated herein by reference in their entirety. The antibodies useful for the present invention can alternatively be glycoengineered to have reduced fucose residues in the Fc region according to the techniques disclosed in U.S. Pat. Appl. Pub. No. 2003/0157108 (Genentech), or in EP 1 176 195 A1, WO 03/084570, WO 03/085119 and U.S. Pat. Appl. Pub. Nos. 2003/0115614, 2004/093621, 2004/110282, 2004/110704, 2004/132140, Niwa et al., J Immunol Methods 306, 151/160 (2006), U.S. Pat. No. 6,946,292 (Kyowa). Glycoengineered antibodies useful for the invention may also be produced in expression systems that produce modified glycoproteins, such as those taught in U.S. Pat. Appl. Pub. No. 60/344,169 and WO 03/056914 (GlycoFi, Inc.) or in WO 2004/057002 and WO 2004/024927 (Greenovation).

As used herein, the term “Fc-mediated cellular cytotoxicity” includes antibody-dependent cell-mediated (sometimes also termed “cellular”) cytotoxicity (ADCC) and cellular cytotoxicity mediated by a soluble Fc-fusion protein containing a human Fc-region. It is an immune mechanism leading to the lysis of “antibody-targeted cells” by “human immune effector cells”, wherein:

The “human immune effector cells” are a population of leukocytes that display Fc receptors on their surface through which they bind to the Fc-region of antibodies or of Fc-fusion proteins and perform effector functions. Such a population may include, but is not limited to, peripheral blood mononuclear cells (PBMC) and/or natural killer (NK) cells.

The “antibody-targeted cells” are cells bound by the antibodies or Fc-fusion proteins. The antibodies or Fc fusion-proteins bind to target cells via the protein part N-terminal to the Fc region.

As used herein, the term “increased Fc-mediated cellular cytotoxicity” is defined as either an increase in the number of “antibody-targeted cells” that are lysed in a given time, at a given concentration of antibody or of Fc-fusion protein, in the medium surrounding the target cells, by the mechanism of Fc-mediated cellular cytotoxicity defined above, and/or a reduction in the concentration of antibody, or of Fc-fusion protein, in the medium surrounding the target cells, required to achieve the lysis of a given number of “antibody-targeted cells”, in a given time, by the mechanism of Fc-mediated cellular cytotoxicity. The increase in Fc-mediated cellular cytotoxicity is relative to the cellular cytotoxicity mediated by the same antibody, or Fc-fusion protein, produced by the same type of host cells, using the same standard production, purification, formulation and storage methods, which are known to those skilled in the art, but that has not been produced by host cells that have been glycoengineered, for example engineered to express the glycosyltransferase GnTIII, by the methods described herein. Increases in Fc-mediated cellular cytotoxicity, e.g. ADCC or other effector functions and/or Fc receptor binding of the antibodies useful for the present invention can also achieved by methods other than glycoengineering, for example by amino acid modifications in the Fc region of the antibodies as described below. Combinations of different approaches are also encompassed by the present invention. In certain embodiments, one or more amino acid modifications may be introduced into the Fc region of an antibody useful for the present invention, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions. Certain antibody variants with improved or diminished binding to FcRs are described (see, e.g., U.S. Patent No. 6,737,056; WO 2004/056312, and Shields et al., J Biol Chem 9(2): 6591-6604 (2001)). In certain embodiments, an antibody useful for the invention comprises an Fc region with one or more amino acid substitutions which improve ADCC, e.g., substitutions at positions 298, 333, and/or 334 of the Fc region (EU numbering of residues). For further examples concerning Fc region variants see also U.S. Pat. Appl. Nos. 60/439,498; 60/456,041; 60/514,549; or WO 2004/063351 (variant Fc regions with increased binding affinity due to amino acid modification); or U.S. patent application Ser. No. 10/672,280 or WO 2004/099249 (Fc variants with altered binding to FcγR due to amino acid modification), Duncan & Winter, Nature 322:738-40 (1988); U.S. Pat. No. 5,648,260; U.S. Pat. No. 5,624,821; and WO 94/29351. Fc receptor binding assays can be performed to confirm increased Fc receptor binding.

The term “modified” antibody when used in the context of increasing effector functions, Fc-mediated cytotoxicity, ADCC and the like encompasses glycoengineered antibodies as well as Fc region variant antibodies and antibodies modified in any other way suitable to increase said functions. Combinations of different approaches are also encompassed by the present invention.

By “antibody having increased antibody dependent cell-mediated cytotoxicity (ADCC)” is meant an antibody, as that term is defined herein, having increased ADCC as determined by any suitable method known to those of ordinary skill in the art. One accepted in vitro ADCC assay is as follows:
1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
   i) the PBMCs are isolated using standard density centrifugation procedures and are suspended at 5×10⁶ cells/ml in RPMI cell culture medium;
   ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;
   iii) 100 microliters of the final target cell suspension, prepared as described above, are transferred to each well of a 96-well microtiter plate;
   iv) the antibody is serially diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
   v) for the maximum release (MR) controls, 3 additional wells in the plate containing the labeled target cells, receive 50 microliters of a 2% (v/v) aqueous solution of non-ionic detergent (Nonidet, Sigma, St. Louis), instead of the antibody solution (point iv above);
   vi) for the spontaneous release (SR) controls, 3 additional wells in the plate containing the labeled target cells, receive 50 microliters of RPMI cell culture medium instead of the antibody solution (point iv above);
   vii) the 96-well microtiter plate is then centrifuged at 50×g for 1 minute and incubated for 1 hour at 4° C.;
   viii) 50 microliters of the PBMC suspension (point i above) are added to each well to yield an effector:target cell ratio of 25:1 and the plates are placed in an incubator under 5% CO₂ atmosphere at 37° C. for 4 hours;
   ix) the cell-free supernatant from each well is harvested and the experimentally released radioactivity (ER) is quantified using a gamma counter;
   x) the percentage of specific lysis is calculated for each antibody concentration according to the formula (ER-MR)/(MR-SR) x 100, where ER is the average radioactivity quantified (see point ix above) for that antibody concentration, MR is the average radioactivity quantified (see point ix above) for the MR controls (see point v above), and SR is the average radioactivity quantified (see point ix above) for the SR controls (see point vi above);
4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. The increase in ADCC is relative to the ADCC, measured with the above assay, mediated by the same antibody, produced by the same type of host cells, using the same standard production, purification, formulation and storage methods, which are known to those skilled in the art, but that has not been produced by host cells engineered to overexpress GnTIII.

Other in vitro and/or in vivo cytotoxicity assays can be conducted to confirm the increase of effector functions such as ADCC. Non-limiting examples of in vitro assays to assess ADCC activity of a molecule of interest is described in U.S. Pat. No. 5,500,362 (see, e.g. Hellstrom et al. Proc Natl Acad Sci USA 83: 7059-7063 (1986)) and Hellstrom et al., Proc Natl Acad Sci USA 82: 1499-1502 (1985); U.S. Pat. No. 5,821,337 (see Bruggemann et al., J Exp Med 166: 1351-1361 (1987)). Alternatively, non-radioactive methods may be employed (see, for example, ACTI™ non-radioactive cytotoxicity assay for flow cytometry (CellTechnology Inc., Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of a molecule of interest may be assessed in vivo, e.g., in a animal model such as that disclosed in Clynes et al., Proc Natl Acad Sci USA 95: 652-656 (1998).

As used herein, the term “variant” (or “analog”) refers to a polypeptide differing from a specifically recited polypeptide of the invention by amino acid insertions, deletions, and substitutions, created using, e.g., recombinant DNA techniques. Variants of the antibodies of the present invention include chimeric, primatized or humanized antibodies wherein one or several of the amino acid residues are modified by substitution, addition and/or deletion in such manner that does not substantially affect antigen (e.g. EGFR) binding affinity. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are preferably in the range of about 1 to 20 amino acids, more preferably 1 to 10 amino acids. The variation allowed may be experimentally determined by systematically making insertions, deletions, or substitutions of amino acids in a polypeptide molecule using recombinant DNA techniques and assaying the resulting recombinant variants for activity.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98% or 99% identical to a reference polypeptide can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp App Biosci 6, 237-245 (1990). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence), so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to be made for the purposes of the present invention.

As used herein, the term “EGFR” refers to the human epidermal growth factor receptor (also known as HER-1 or ErbB-1) (Ulrich et al., Nature 309, 418-425 (1984); SwissProt Accession #P00533; secondary accession numbers: O00688, O00732, P06268, Q14225, Q68GS5, Q92795, Q9BZS2, Q9GZX1, Q9H2C9, Q9H3C9, Q9UMD7, Q9UMD8, Q9UMG5), as well as naturally-occurring isoforms and variants thereof. Such isoforms and variants include but are not limited to the EGFRvIII variant, alternative splicing products (e.g., as identified by SwissProt Accession numbers P00533-1, P00533-2, P00533-3, P00533-4), variants GLN-98, ARG-266, Lys-521, ILE-674, GLY-962, and PRO-988 (Livingston et al., NIEHS-SNPs, environmental genome project, NIEHS ES15478, Department of Genome Sciences, Seattle, Wash. (2004)), and others identified by the following accession numbers: NM_{—}005228.3, NM_{—}201282.1, NM_{—}201283.1, NM_{—}201284.1 (REFSEQ mRNAs); AF125253.1, AF277897.1, AF288738.1, AI217671.1, AK127817.1, AL598260.1, AU137334.1, AW163038.1, AW295229.1, BCO57802.1, CB160831.1, K03193.1, U48722.1, U95089.1, X00588.1, X00663.1; H5448451, H5448453, H5448452 (MIPS assembly); DT.453606, DT.86855651, DT.95165593, DT.97822681, DT.95165600, DT.100752430, DT.91654361, DT.92034460, DT.92446349, DT.97784849, DT.101978019, DT.418647, DT.86842167, DT.91803457, DT.92446350, DT.95153003, DT.95254161, DT.97816654, DT.87014330, DT.87079224 (DOTS Assembly).

As used herein, the term “IGF-1R” refers to the human insulin-like growth factor receptor-1 (also known as CD221 antigen) (SwissProt Accession #P08069, EC 2.7.10.1 (former EC 2.7.112); LeRoith et al., Endocrin Rev 16, 143-163 (1995); and Adams et al., Cell Mol Life Sci 57, 1050-1093 (2000)) as well as naturally occurring isoforms and variants thereof.

As used herein, the term “binding to IGF-1R” means the binding of the antibody to IGF-1R in an in vitro assay, preferably in a binding assay in which the antibody is bound to a surface and binding of IGF-1R is measured by Surface Plasmon Resonance (SPR). Binding means a binding affinity (K_{D}) of 10⁻⁸ M or less, preferably 10⁻¹³ to 10⁻⁹ M. The binding affinity is determined with a standard binding assay, such as surface plasmon resonance technique (Biacore®).

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

As used herein, the term “ligand” refers to a polypeptide which binds to and/or activates a receptor, such as EGFR or IGF-1R. The term includes membrane-bound precursor forms of the ligand, as well as proteolytically processed soluble forms of the ligand.

As used herein, the term “ligand activation of EGFR/IGF-1R” refers to signal transduction (e.g., that caused by an intracellular kinase domain of EGFR/IGF-1R phosphorylating tyrosine residues in the receptor itself or a substrate polypeptide) mediated by EGFR/IGF-1R ligand binding.

As used herein, the term “disease or disorder characterized by abnormal activation or production of EGFR and/or IGF-1R or an EGFR and/or IGF-1R ligand or disorder related to EGFR and/or IGF-1R expression”, refers to a condition, which may or may not involve malignancy or cancer, where abnormal activation and/or production of EGFR and/or IGF-1R, and/or an EGFR and/or IGF-1R ligand is occurring in cells or tissues of a subject having, or predisposed to, the disease or disorder.

As used herein, the terms “overexpress”, “overexpressed”, and “overexpressing”, as used in connection with cells expressing EGFR and/or IGF-1R, refer to cells which have measurably higher levels of EGFR and/or IGF-1R, respectively, on the surface thereof compared to a normal cell of the same tissue type. Such overexpression may be caused by gene amplification or by increased transcription or translation. EGFR and/or IGF-1R expression (and, hence, overexpression) may be determined in a diagnostic or prognostic assay by evaluating levels of EGFR and/or IGF-1R present on the suface of a cell or in a cell lysate by techniques that are known in the art: e.g., via an immunohistochemistry assay, immunofluorescence assay, immunoenzyme assay, ELISA, flow cytometry, radioimmunoassay, Western blot, ligand binding, kinase activity, etc. (see generally, Cell Biology: A Laboratory Handbook, Celis, J., ed., Academic Press (2nd ed., 1998); Current Protocols in Protein Science, Coligan, J. E. et al., eds., John Wiley & Sons (1995-2003); see also Sumitomo et al., Clin Cancer Res 10, 794-801 (2004), Pantaleo et al., Int J Cancer 125, 2991-2994 (2009) the entire contents of which are herein incorporated by reference). Alternatively, or additionally, one may measure levels of EGFR- and/or IGF-1R- encoding nucleic acid molecules in the cell, e.g., via fluorescent in situ hybridization, Southern blotting, or PCR techniques. The levels of EGFR and/or IGF-1R in normal cells are compared to the levels of cells affected by a cell proliferation disorder (e.g., cancer) to determine if EGFR and/or IGF-1R are overexpressed.

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expression of a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinase expression and/or activation; (5) any tumors that proliferate by aberrant serine/threonine kinase activation; and (6) benign and malignant cells of other proliferative diseases in which aberrant serine/threonine kinase activation occurs.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The patient may be a human or an animal. The term “treatment” as used herein, unless otherwise indicated, refers to the act of treating.

The phrase “a method of treating” or its equivalent, when applied to, for example, cancer refers to a procedure or course of action that is designed to reduce or eliminate the number of cancer cells in a human or animal, prevent the progression of a cancer, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a human or animal, is nevertheless deemed an overall beneficial course of action.

The term “therapeutically effective or therapeutic agent” means a substance or composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

All patents, published patent applications and other references disclosed herein are hereby expressly incorporated herein by reference.

Those skilled in the art will recognize, or be able to ascertain, using no more than routine experimentation, many equivalents to specific aspects of the invention described specifically herein. Such equivalents are intended to be encompassed in the scope of the following claims.

### EXAMPLES

### Material and General Methods

### Antibodies

hu-ICR62 IgG1 anti-EGFR mAb and rhu anti-IGF-1R mAb 18 are manufactured by techniques generally known from the production of recombinant proteins. Techniques to manufacture hu-ICR62 IgG1 anti-EGFR mAb are described in WO 2006/082515 and WO 2008/017963, techniques to manufacture rhu anti-IGF-1R mAb 18 in EP1646720, WO 2007/115814 and WO 2008/077546. Briefly, genetically engineered Chinese hamster ovary cell lines (CHO) are expanded in cell culture from a master cell bank. The antibodies are purified from the conditioned cell culture medium using protein A affinity chromatography on a Mab Select SuRe™ column (GE), followed by cation exchange chromatography on a Capto S™ column (GE) (hu-ICR62 IgG1 anti-EGFR mAb) or ceramic hydroxyapatite chromatography on a CHT™ column (BioRad) (rhu anti-IGF-1R mAb 18), and a final anion exchange chromatographic step on a Capto Q™ column (GE) (hu-ICR62 IgG1 anti-EGFR mAb) or a Q-Sepharose FF™ column (GE) (rhu anti-IGF-1R mAb 18). Viruses are removed by nanofiltration using a Viresolve® Pro membrane (Millipore) and the antibodies are concentrated and transferred into the desired buffer by diafiltration.

For the manufacture of partially fucosylated antibodies, CHO cell lines overexpressing β(1,4)-N-acetylglucosaminyltransferase III (GnTIII) and α-mannosidase II (ManII) are used, as described in U.S. Pat. No. 7,517,670, and specifically described in WO 2006/082515 and WO 2008/017963 (for hu-ICR62 IgG1 anti-EGFR mAb) and in WO 2008/077546 (for rhu anti-IGF-1R mAb 18).

Cetuximab (Erbitux™) was obtained from Merck.

Cell Culture

A549 human lung adenocarcinoma cells (NSCLC), originally obtained from ATCC, were routinely cultured in Dulbecco's Modified Eagle Medium (DMEM) (Gibco, Switzerland) supplemented with 10% fetal bovine serum (Invitrogen, Switzerland) and 2 mM L-glutamine (Gibco, Switzerland) at 37° C. in a water-saturated atmosphere at 5% CO₂. Culture passage was performed every third day, using trypsin/ethylenediaminetetraacetic acid (EDTA) 1× solution (Gibco, Switzerland).

Animals

SCID beige mice; age 6-7 weeks at arrival; mean body weight >20 g; (purchased from Charles River, Germany) were maintained under specific-pathogen-free conditions with daily cycles of 12 h light/12 h darkness according to committed guidelines (GV-Solas; Felasa; TierschG I). Experimental study protocol was reviewed and approved by local government; registration no. P200586. After arrival animals were maintained in the quarantine part of the animal facility for one week to get accustomed to the new environment and for observation. Continuous health monitoring was carried out on regular basis. Diet food (Provimi Kliba 3337) and water (acidified pH 2.5-3) were provided ad libitum.

Tumor Cell Injection

On the day of injection, A549 tumor cells were harvested from culture flasks (Greiner Bio-One) using trypsin-EDTA (Gibco, Switzerland), transferred into 50 ml culture medium, washed once and resuspended in AIM V medium (Gibco, Switzerland). After an additional wash with AIM V, cell concentration was determined using a cell counter. For injection of A549 cells, the final titer was adjusted to 5.0×10⁶ cells/ml. Subsequently 200 μl of this mixture was injected into the lateral tail vein of the mice using a 1.0 ml tuberculin syringe (BD Biosciences, Germany).

Identification/Staging

Mice were randomly distributed at staging. Animals were housed in M3 size cages. Individual identification of the animals was assured by marking the animals with a permanent dye.

### Example 1

### Survival of Mice Bearing Lung Adenocarcinoma Xenografts, Treated with Combinations of Anti-EGFR Antibodies and Anti-IGF-1R Antibody

The in vivo anti-tumor efficacy of combining rhu anti-IGF-1R mAb 18 with the partially fucosylated hu-ICR62 IgG1 anti-EGFR mAb, compared to the combination of anti-IGF1R with the commercially available anti-EGFR antibody cetuximab, was analyzed in the A549 lung adenocarcinoma xenograft model. The primary parameter was survival.

General materials and methods were as described above. Specifically, A549 cells at culture passage 10 were injected into SCID beige mice as described above. The antibody treatment was started at the time of staging, 14 days after cell injection. 10 mice were used per group.

The animals were treated either with the corresponding vehicle alone, with 25 mg/kg partially fucosylated hu-ICR62 IgG1 anti-EGFR mAb, with 25 mg/kg partially fucosylated hu-ICR62 IgG1 anti-EGFR mAb and 10 mg/kg rhu anti-IGF-1R mAb 18, or with 25 mg/kg cetuximab (Erbitux™) and 10 mg/kg rhu anti-IGF-1R mAb 18. The antibodies and the corresponding vehicle were administered intraperitoneally (i.p.) once weekly for 3 weeks. The antibodies were prepared freshly from stock before use.

Animals were controlled daily for clinical symptoms and detection of adverse effects namely, respiratory distress, impaired motility and scruffy fur. Termination criteria and study exclusion criteria for animals were described and approved in the corresponding project license. Animals were sacrificed according to the termination criteria (impaired locomotion, scruffy fur and arched back observed for two consecutive days).

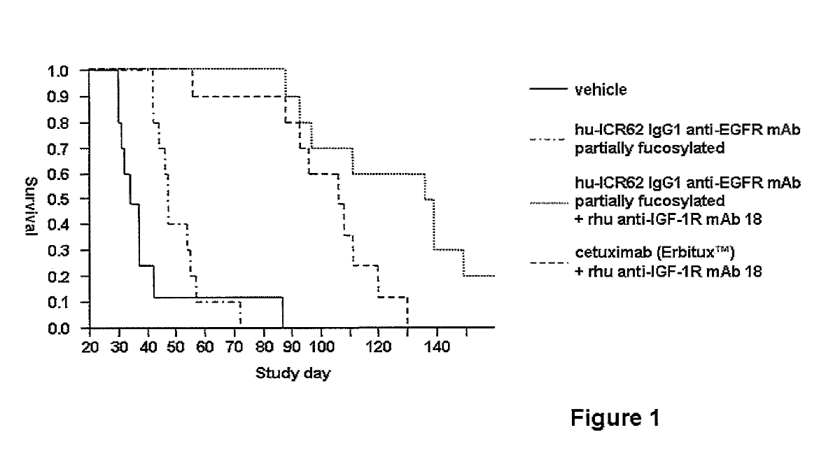

Survival data are shown as Kaplan-Meier curves in FIG. 1. The data was statistically analyzed by pairwise log-rank test. It was found that the combination of partially fucosylated hu-ICR62 IgG1 anti-EGFR mAb and rhu anti-IGF-1R mAb 18 significantly prolonged survival compared to the combination of cetuximab with the same anti-IGF-1R antibody (median survival 139 days vs. 106 days; p=0.019), and that both combinations significantly prolonged survival compared to the vehicle (median survival 34 days) or the hu-ICR62 IgG1 anti-EGFR mAb alone (median survival 47 days).

### Example 2

### Survival of Mice Bearing Lung Adenocarcinoma Xenografts, Treated with a Combination of Anti-EGFR Antibody and Partially Fucosylated Anti-IGF-1R Antibody

The in vivo anti-tumor efficacy of combining partially fucosylated rhu anti-IGF-1R mAb 18 with the partially fucosylated hu-ICR62 IgG1 anti-EGFR mAb, compared to either of these two antibodies alone, was analyzed in the A549 lung adenocarcinoma xenograft model. The primary parameter was survival.

General materials and methods were as described above. Specifically, A549 cells at culture passage 12 were transplanted into SCID beige mice as described above. The antibody treatment was started at the time of staging, 10 days after cell injection. 10 mice were used per group.

The animals were treated either with the corresponding vehicle alone, with 25 mg/kg partially fucosylated hu-ICR62 IgG1 anti-EGFR mAb, with 10 mg/kg partially fucosylated rhu anti-IGF-1R mAb 18, or with 25 mg/kg partially fucosylated hu-ICR62 IgG1 anti-EGFR mAb and 10 mg/kg rhu anti-IGF-1R mAb 18. The antibodies and the corresponding vehicle were administered intraperitoneally (i.p.) once weekly for 3 weeks. The antibodies were prepared freshly from stock before use.

Animals were controlled daily for clinical symptoms and detection of adverse effects namely, respiratory distress, impaired motility and scruffy fur. Termination criteria and study exclusion criteria for animals were described and approved in the corresponding project license. Animals were sacrificed according to the termination criteria (impaired locomotion, scruffy fur and arched back observed for two consecutive days).

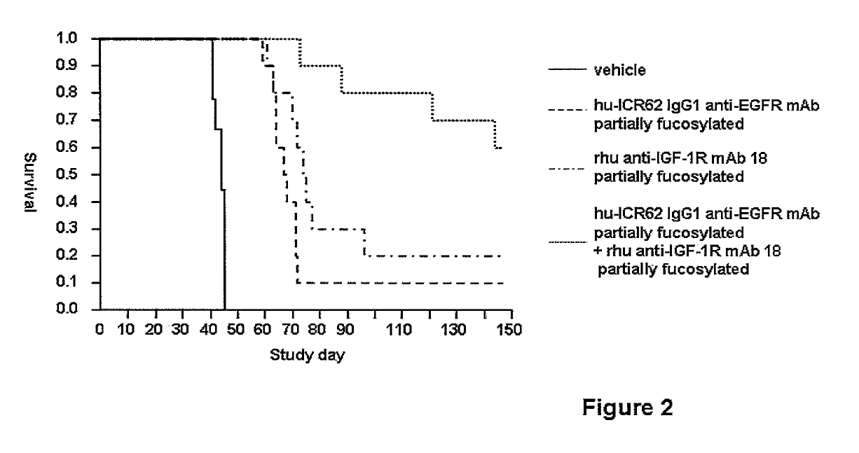

Survival data are shown as Kaplan-Meier curves in FIG. 2. The data was statistically analyzed by pairwise log-rank test (Table 6). It was found that the combination of partially fucosylated hu-ICR62 IgG1 anti-EGFR mAb and partially fucosylated rhu anti-IGF-1R mAb 18 significantly prolonged survival compared to the vehicle or either antibody alone. Median survival was 34 days for vehicle-treated animals, 58 days or 65 days for the animals treated with partially fucosylated hu-ICR62 IgG1 anti-EGFR mAb or partially fucosylated rhu anti-IGF-1R mAb 18, respectively, while 60% of the animals treated with the combination of the two antibodies survived until the study was terminated at 147 days.

TABLE 6
Statistical analysis of anti-tumor activity: Pairwise log-rank test.

| | | | | Partially |
|---|---|---|---|---|
| | | | | fucosylated |
| | | Partially | | hu-ICR62 |
| | | fucosylated | | IgG1 anti-EGFR |
| | | hu-ICR62 | Partially | mAb + partially |
| | | IgG1 anti- | fucosylated | fucosylated rhu |
| | | EGFR | rhu anti-IGF- | anti-IGF-1R |
| Group | Vehicle | mAb | 1R mAb 18 | mAb 18 |
| Vehicle | 1.0 | <0.0001 | <0.0001 | <0.0001 |
| Partially | <0.0001 | 1.0 | 0.1134 | 0.0005 |
| fucosylated | | | | |
| hu-ICR62 | | | | |
| IgG1 anti- | | | | |
| EGFR mAb | | | | |
| Partially | <0.0001 | 0.1134 | 1.0 | 0.0195 |
| fucosylated | | | | |
| rhu anti-IGF- | | | | |
| 1R mAb 18 | | | | |
| Partially | <0.0001 | 0.0005 | 0.0195 | 1.0 |
| fucosylated | | | | |
| hu-ICR62 IgG1 | | | | |
| anti-EGFR | | | | |
| mAb + | | | | |
| partially | | | | |
| fucosylated | | | | |
| rhu anti-IGF- | | | | |
| 1R mAb 18 | | | | |

1. A combination comprising a humanized IgG-class anti-EGFR antibody and an anti-IGF-1R antibody, wherein the IgG-class anti-EGFR antibody comprises
a) in the heavy chain variable domain a CDR1 of SEQ ID NO:1, a CDR2 of SEQ ID NO:16 and a CDR3 of SEQ ID NO:31, and
b) in the light chain variable domain a CDR1 of SEQ ID NO:33, a CDR2 of SEQ ID NO:34 and a CDR3 of SEQ ID No:35.

2. The combination of claim 1, wherein the humanized IgG-class anti-EGFR antibody is glycoengineered to have an altered oligosaccharide structure in the Fc region.

3. The combination of claim 2, wherein the humanized IgG-class anti-EGFR antibody has an increased proportion of non-fucosylated oligosaccharides in its Fc-region, compared to a non-glycoengineered antibody.

4. The combination of claim 1, wherein the anti-IGF-1R antibody is modified to have increased effector function and/or increased Fc receptor binding compared to an unmodified antibody.

5. The combination of claim 4, wherein the anti-IGF-1R antibody is glycoengineered to have an altered oligosaccharide structure in the Fc region.

6. The combination claim 1 or 5, wherein the anti-IGF-1R antibody comprises a heavy chain variable domain amino acid sequence of SEQ ID NO:41 or SEQ ID NO:43 and a light chain variable domain amino acid sequence of SEQ ID NO:42 or SEQ ID NO:44.

7. The combination of claim 1, wherein the humanized IgG-class anti-EGFR antibody and the anti-IGF-1R antibody are contained in the same formulation.

8. The combination of claim 1, wherein the humanized IgG-class anti-EGFR antibody and the anti-IGF-1R antibody are contained in different formulations.

9. A pharmaceutical composition comprising a humanized IgG-class anti-EGFR antibody and an anti-IGF-1R antibody in a pharmaceutically acceptable carrier, wherein the IgG-class anti-EGFR antibody comprises
a) in the heavy chain variable domain a CDR1 of SEQ ID NO:1, a CDR2 of SEQ ID NO:16 and a CDR3 of SEQ ID NO:31, and
b) in the light chain variable domain a CDR1 of SEQ ID NO:33, a CDR2 of SEQ ID NO:34 and a CDR3 of SEQ ID No:35.

10. The pharmaceutical composition of claim 9, wherein the humanized IgG-class anti-EGFR antibody is glycoengineered to have an altered oligosaccharide structure in the Fc region.

11. The pharmaceutical composition of claim 9 or 10, wherein the anti-IGF-1R antibody is modified to have increased effector function and/or increased Fc receptor binding compared to an unmodified antibody.

12. The pharmaceutical composition of claim 9, wherein the composition additionally comprises one or more other therapeutically active agents.

13. A method for the treatment of cancer, comprising administering to a subject in need thereof a humanized IgG-class anti-EGFR antibody and an anti-IGF-1R antibody, wherein the IgG-class anti-EGFR antibody comprises
a) in the heavy chain variable domain a CDR1 of SEQ ID NO:1, a CDR2 of SEQ ID NO:16 and a CDR3 of SEQ ID NO:31, and
b) in the light chain variable domain a CDR1 of SEQ ID NO:33, a CDR2 of SEQ ID NO:34 and a CDR3 of SEQ ID No:35.

14. The method of claim 13, wherein the humanized IgG-class anti-EGFR antibody is glycoengineered to have an altered oligosaccharide structure in the Fc region.

15. The method of claim 13 or 14, wherein the anti-IGF-1R antibody is modified to have increased effector function and/or increased Fc receptor binding compared to an unmodified antibody.

16. The method of claim 13, wherein the humanized IgG-class anti-EGFR antibody and the anti-IGF-1R antibody are administered in the same formulation.

17. The method of claim 13, wherein the humanized IgG-class anti-EGFR antibody and the anti-IGF-1R antibody are administered in different formulations.

18. The method of claim 13, wherein the humanized IgG-class anti-EGFR antibody and the anti-IGF-1R antibody are administered by the same route.

19. The method of claim 13, wherein the humanized IgG-class anti-EGFR antibody and the anti-IGF-1R antibody are administered by different routes.

20. The method of claim 13, further comprising administering one or more anti-cancer agents to the subject.

21. A method for manufacturing a medicament, comprising combining a therapeutically effective amount of a humanized IgG-class anti-EGFR antibody and an anti-IGF-1R antibody, wherein the IgG-class anti-EGFR antibody comprises
a) in the heavy chain variable domain a CDR1 of SEQ ID NO:1, a CDR2 of SEQ ID NO:16 and a CDR3 of SEQ ID NO:31, and
b) in the light chain variable domain a CDR1 of SEQ ID NO:33, a CDR2 of SEQ ID NO:34 and a CDR3 of SEQ ID No:35.

22. A kit comprising a humanized IgG-class anti-EGFR antibody and an anti-IGF-1R antibody, in the same or in separate containers, wherein the IgG-class anti-EGFR antibody comprises
a) in the heavy chain variable domain a CDR1 of SEQ ID NO:1, a CDR2 of SEQ ID NO:16 and a CDR3 of SEQ ID NO:31, and
b) in the light chain variable domain a CDR1 of SEQ ID NO:33, a CDR2 of SEQ ID NO:34 and a CDR3 of SEQ ID No:35.
